SEQUENCE LISTING (5 sequences)

SEQ ID NO: 1 - DNA, 21 bp, synthetic construct
  source 1..21: mol_type = other DNA
atccacagtcagccataataa

SEQ ID NO: 2 - DNA, 21 bp, synthetic construct
  source 1..21: mol_type = other DNA
attcctgtcctccgttacctg

SEQ ID NO: 3 - DNA, 20 bp, synthetic construct
  source 1..20: mol_type = other DNA
gtggcccttctcaaatgtcc

SEQ ID NO: 4 - DNA, 19 bp, synthetic construct
  source 1..19: mol_type = other DNA
cccaacccttccctgaatg

SEQ ID NO: 5 - DNA, 21 bp, synthetic construct
  source 1..21: mol_type = other DNA
ttctcctgacagtgctcaatc

(19) United States
(12) Patent Application Publication
(10) Pub. No.: US 2025/0059139 A1
(43) Pub. Date: Feb. 20, 2025

(54) COMPOUNDS FOR TREATMENT OF CANCER

(71) Applicant: THE JOHNS HOPKINS UNIVERSITY, Baltimore, MD (US)

(72) Inventors: John T. Isaacs, Baltimore, MD (US); William Nathaniel Brennen, Baltimore, MD (US); Emmanuel S. Akinboye, Baltimore, MD (US)

(21) Appl. No.: 18/721,824

(22) PCT Filed: Dec. 20, 2022
(86) PCT No.: PCT/US22/82050

§ 371 Date: Jun. 19, 2024

Related U.S. Application Data
(60) Provisional application No. 63/291,757, filed on Dec. 20, 2021.

(51) Int. Cl. C07D 215/56; A61K 31/337; A61K 31/4704; A61P 35/00

(52) U.S. Cl. CPC C07D 215/56; A61K 31/337; A61K 31/4704; A61P 35/00

(57) ABSTRACT

The present disclosure relates to compounds that can be used in the treatment of cancer, such as prostate cancer. The disclosure also relates to pharmaceutical compositions comprising the prodrugs, and related methods of treatment.

### CROSS-REFERENCE TO RELATED APPLICATIONS

This application claims priority to U.S. Provisional Patent Application No. 63/291,757, filed on Dec. 20, 2021, which is hereby incorporated by reference herein in its entirety.

### STATEMENT REGARDING FEDERALLY SPONSORED RESEARCH

This invention was made with government support under grants W81XWH-16-1-0410, W81XWH-18-1-0348, and W81XWH-17-1-0528 awarded by the Department of Defense, and grants CA006973, CA058236, and CA255259 awarded by the National Institutes of Health. The government has certain rights in the invention.

### FIELD

The present disclosure relates to compounds that can be used in the treatment of cancer, such as prostate cancer. The disclosure also relates to pharmaceutical compositions comprising the compounds, and related methods of treatment.

### BACKGROUND

Prostate cancer is the most commonly diagnosed cancer in men in the United States. It remains an incurable disease once progression to the metastatic castration-resistant (mCRPC) state occurs. Unfortunately, each of the FDA-approved agents for mCRPC produces only modest increases in overall survival followed by the emergence of resistance and a more aggressive phenotype.

Linomide, also known as roquinimex, is a first-generation oral quinoline-3-carboxamide. In preclinical studies, Linomide showed robust efficacy against solid malignances, particularly metastatic castration-resistant prostate cancer (mCRPC) via its antiangiogenic, immunomodulatory, and anti-metastatic properties. However, clinical development of Linomide was halted after Phase III clinical trials resulted in several cases of pericarditis and neuropathy (Noseworthy et al. Neurology 2000, 54:1726-1733; Tan et al. Mult. Scler. 2000, 6:99-104).

Tasquinimod (TasQ) was identified as a second-generation oral quinoline-3-carboxamide. In preclinical studies, it was shown to have a 30-60 fold enhanced potency in antiangiogenic and anti-metastatic ability against solid malignances, particularly metastatic castration-resistant prostate cancer (mCRPC). In a Phase III clinical trial, a daily TasQ oral dose of 1 mg/day was found to significantly reduce the risk of radiographic progression or death vs. placebo by 36% in mCRPC patients progressing on maintenance Androgen Depreciation Therapy (ADT); however at this 1 mg/day oral dose, overall survival was not enhanced (Sternberg et al. J. Clin. Oncol. 2016, 34(22):2636-43.

### SUMMARY

Provided herein are compounds of formula (I):

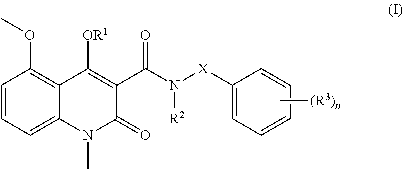

or a pharmaceutically acceptable salt thereof, wherein:
R¹ is selected from hydrogen and C₁-C₄ alkyl, C₁-C₄ hydroxyalkyl, and C₁-C₄ aminoalkyl, wherein the amino of the C₁-C₄ aminoalkyl is optionally protected by a protecting group;
R² is selected from hydrogen, C₃-C₁₀ alkyl, C₃-C₆, cycloalkyl, and arylalkyl, wherein the alkyl, cycloalkyl, and arylalkyl are each optionally substituted with 1, 2, or 3 substituents independently selected from halo, C₁-C₄ haloalkyl, hydroxy, C₁-C₄ alkoxy, and arylalkyloxy;
n is 0, 1, 2, or 3;
each R³ is independently selected from C₁-C₄ haloalkyl, halo, C₁-C₄ alkyl, and C₁-C₄ alkoxy; and
X is a bond or —N═CH—, wherein if X is a bond, R² is not hydrogen.

In some embodiments, R¹ is selected from hydrogen, methyl, ethyl, and 2-aminoethyl, wherein the amino of the 2-aminoethyl is optionally protected by a tert-butyloxycarbonyl group. In some embodiments, R¹ is hydrogen.

In some embodiments, X is a bond, and R² is selected from C₃-C₁₀ alkyl, C₃-C₆ cycloalkyl, and arylalkyl, wherein the alkyl, cycloalkyl, and arylalkyl are each optionally substituted with 1, 2, or 3 substituents independently selected from halo, C₁-C₄ haloalkyl, hydroxy, C₁-C₄ alkoxy, and arylalkyloxy. In some embodiments, X is a bond, and R² is selected from isopropyl, n-heptyl, cyclohexyl, benzyl, and ethyl substituted with one benzyloxy group.

In some embodiments, X is —N═CH— and R² is hydrogen.

In some embodiments, n is 0. In some embodiments, n is 1. In some embodiments, R³ is substituted at the para position of the phenyl group. In some embodiments, R³ is C₁-C₄ haloalkyl. In some embodiments, R³ is trifluoromethyl.

In some embodiments, the compound is a compound of formula (Ia):

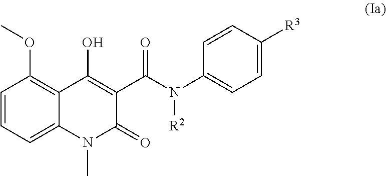

or a pharmaceutically acceptable salt thereof, wherein:
R² is selected from C₄-C₆ alkyl and C₃-C₆ cycloalkyl; and
R³ is independently selected from C₁-C₄ haloalkyl, halo, C₁-C₄ alkyl, and C₁-C₄ alkoxy.

In some embodiments, R² is C₃-C₆, cycloalkyl, and R³ is C₁-C₄ haloalkyl.

In some embodiments, the compound is selected from:

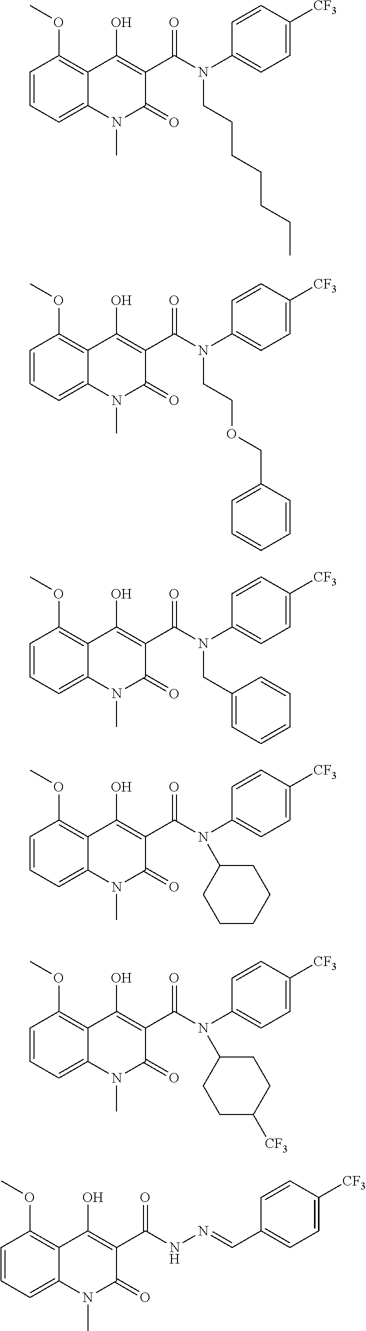

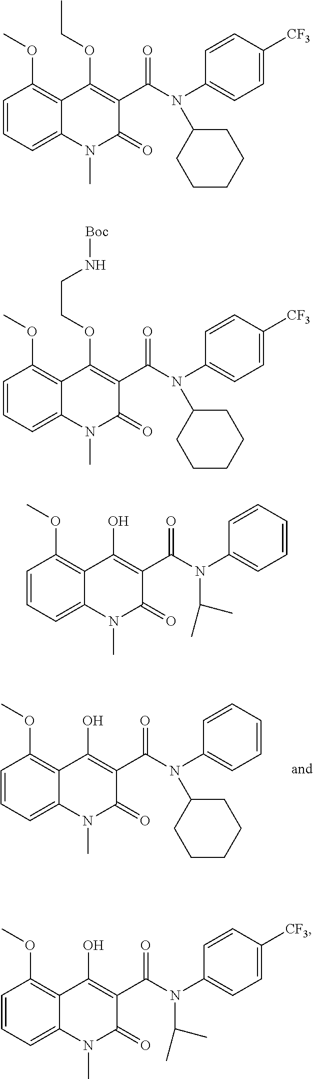 [SMILES: CCOc1c(C(=O)N(c2ccc(C(F)(F)F)cc2)C2CCCCC2)c(=O)n(C)c2cccc(OC)c12 ; COc1cccc2c1c(OCCNC(=O)OC(C)(C)C)c(C(=O)N(c1ccc(C(F)(F)F)cc1)C1CCCCC1)c(=O)n2C ; COc1cccc2c1c(O)c(C(=O)N(c1ccccc1)C(C)C)c(=O)n2C ; COc1cccc2c1c(O)c(C(=O)N(c1ccccc1)C1CCCCC1)c(=O)n2C ; COc1cccc2c1c(O)c(C(=O)N(c1ccc(C(F)(F)F)cc1)C(C)C)c(=O)n2C]

or a pharmaceutically acceptable salt thereof.

Also disclosed herein is a pharmaceutical composition comprising a compound disclosed herein (e.g., a compound of formula (I)), and a pharmaceutically acceptable carrier.

Also disclosed herein is a method of treating cancer in a subject in need thereof, comprising administering to the subject a therapeutically effective amount of a compound disclosed herein (e.g., a compound of formula (I)), or a pharmaceutical composition disclosed herein (e.g., a pharmaceutical composition comprising a compound of formula (I)). In some embodiments, the cancer is prostate cancer. In some embodiments, the prostate cancer is metastatic castration-resistant prostate cancer. In some embodiments, the method further comprises administering an additional chemotherapeutic agent to the subject. In some embodiments, the additional chemotherapeutic agent is a taxane. In some embodiments, the subject is a human.

Also disclosed herein is the use of a compound disclosed herein (e.g., a compound of formula (I)), or a pharmaceutical composition disclosed herein (e.g., a pharmaceutical composition comprising a compound of formula (I)), in the treatment of cancer. In some embodiments, the cancer is prostate cancer. In some embodiments, the prostate cancer is metastatic castration-resistant prostate cancer.

### BRIEF DESCRIPTION OF THE DRAWINGS

FIG. 1 shows dose-response inhibition of in vivo growth of CWR22-RH human prostate cancer PDXs in castrate male NSG mice (n=5/group) over a 3-week treatment period with the indicated oral daily dose of TasQ initiated when xenografts were ˜0.2 cc (left-side Y-axis); and TasQ serum Cₘₐₓ level at indicated dose (right-side Y-axis).

FIG. 2 shows a summary of % PAL AHR binding inhibited by indicated concentration of TasQ.

FIG. 3 shows real time (RT)-PCR analysis of CYP1A1 mRNA level in LNCaP cells (n=3) treated for 12 hrs with increasing concentrations of TasQ.

FIG. 4 shows Western immunoblot (IB) analysis of the cytoplasmic (C) vs. nuclear (N) level of AHR protein in LNCaP human prostate cancer cells treated for 30 minutes with increasing TasQ concentrations. 100,000 cells loaded/lane.

FIG. 5 shows a time course of change in the cytoplasmic (C) vs. nuclear (N) level of AHR protein in LNCaP cells treated with TasQ (10 μM).

FIG. 6 shows relative expression of mouse Liver Cyp1A1 (mCYP1A1) in animals (n=3) 24 hrs after receiving indicated oral dose of TasQ normalized to animals receiving only vehicle. 100,000 cells loaded/lane.

FIG. 7 shows growth response of LvCaP-3 human prostate cancer PDXs in castrate mice to daily oral dosing with TasQ (10 mg/kg/d) or Benzo[a]pyrene [B(a)P]alone or in combination initiated when xenografts were ˜0.2 cc. (n=5/group).

FIG. 8 shows initial growth response of LNCaP parental control vs. AHR KD cells in castrate mice (n=5/group) and their subsequent response to oral dosing with TasQ (10 mg/kg/d). Insert: 1B of AHR protein level in control vs. AHR KD LNCaP cells. Arrow indicates time of initiation of TasQ treatment.

FIG. 9 shows growth response of TRAMP-C2 mouse prostate cancer in C57BL/6 wt vs. NADPH oxidase Knockout (KO), iNOS Knockout (KO), or S100A9 Knockout (KO) intact male mice to daily oral dosing with TasQ [10 mg/kg/d, (n=5/group)].

FIG. 10 shows growth response of B6CaP mouse prostate cancer in C57BL/6 wt vs. S100A9-null intact male mice to daily oral dosing with TasQ [10 mg/kg/d, (n=5/group)].

FIG. 11 shows dose response binding to immobilized amine-linked TasQ obtained after injection of 62.5 to 500 nM fl-HDAC4 in HBS-P buffer containing ZnCl₂ (10 μM).

FIG. 12 shows steady-state dose response binding of fl-HDAC4 indicating saturable binding to immobilized TasQ.

FIG. 13 shows steady-state dose response binding of TasQ vs. Linomide to immobilized fl-HDAC4.

FIG. 14 shows steady-state dose response binding of fl-HDAC4 (AA 1-972), but not AA 672-972 or AA 551-648 truncated HDAC4, to immobilized TasQ.

FIG. 15 shows dose-response effect of TasQ, Trichostatin A, or TFMK on HDAC4 enzymatic activity assayed using trifluoroacetamide substrate (aka Lys(Tfa)-AMC).

FIG. 16 shows steady-state binding of fl-HDAC4 to immobilized TasQ is not inhibited by “locking” HDAC4 in the open confirmation due to its binding of increasing concentration of TFMK.

FIG. 17 shows computer-based docking of TasQ to inactive (non-NCoR binding) conformation of regulatory zinc-binding domain (ZRD) within the catalytic domain (amino acids 648-1051) of human HDAC4. Magenta colored ball is Zn²⁺ in the ZRD. In the right panel is an enlargement to indicate the position of R681 and R798.

FIG. 18 shows inhibition of wildtype (wt) vs. R681A/R798A mutant HDAC4 binding to immobilized TasQ after pre-incubation with 0.1-500 μM TasQ in solution.

FIG. 19 shows dose response binding of fl-wt HDAC4 obtained after pre-incubation with TasQ (1-100 μM) to immobilized fl-wt NCoR1 in the presence of ZnCl₂ (10 μM).

FIG. 20 shows inhibition of HDAC4-NCoR1 binding by TasQ expressed as percent (%) reduction in steady-state SPR response.

FIG. 21 shows SPR response detected binding of 50 nM wt fl-HDAC4 (upper sensorgram) vs. R681A/R798A mutant fl-HDAC4 (lower sensorgram) to immobilized TasQ.

FIG. 22 shows dose response growth inhibition by TasQ of wt parental vs. HDAC4 KD LNCaP cells in normoxia vs. hypoxia.

FIG. 23 shows IB of HDAC4 protein in LN-95 parental control cells vs. HDAC4 KD clones and densitometry.

FIG. 24 shows in vitro growth of LN-95 parental cells (vs. HDAC4 KD clone 3 (cl. 3) cells (n=8/group).

FIG. 25 shows in vivo growth response (n=5/group) of LN-95 parental xenografts in untreated castrate mice vs. xenografts in castrated mice treated daily with TasQ (10 mg/kg/d) starting on day 25 post-inoculation vs. HDAC4 KD cl. 3 xenograft in untreated castrated mice.

FIG. 26 shows IB of HDAC4 protein level in control vs. HDAC4 KD clones of LAPC-4 cells (note upper band is the sumoylated form, lower band is unsumoylated ˜140 kDa size) and densitometry. 100,000 cells loaded/lane.

FIG. 27 shows in vivo growth response (n=5/group) of LAPC-4 parental vs. HDAC4 KD clones xenografted in untreated intact mice vs. parental LAPC4 xenografts in intact mice treated daily with TasQ (30 mg/kg/d) starting on day 28 post-inoculation.

FIG. 28 shows 1B of NCoR1 protein in parental LNCaP cells transfected with flag-tagged fl-wt vs. H863L dominant-negative (DN) mutant fl-HDAC4 immunoprecipitated (IP) with Flag. 100,000 cells loaded/lane.

FIG. 29 shows inhibition in in vivo growth of LNCaP xenografts expressing H863L-mutant HDAC4 or HIF-1α KD in intact mice expressed as the increase in time post-inoculation to reach 500 mm³ (n=5/group).

FIG. 30 shows IB of HIF-1α protein in LNCaP cells in normoxic vs. hypoxic (i.e., 1% pO₂) in vitro conditions with and without the addition of indicated TasQ concentration. 100,000 cells loaded/lane.

FIG. 31 shows nuclear HIF-1α protein level under hypoxia in untreated wt LNCaP cells vs. treated with TasQ vs. HIF-1a KD or HDAC4 KD cells.

FIG. 32 shows IB of HIF-1α protein in control LNCaP cells in normoxic (N) vs. hypoxic (H) in vitro conditions and in HIF-1α KD clones (cl) under these same conditions. 100,000 cells loaded/lane.

FIG. 33 shows 1B of HIF-1α protein in parental CWR-22Rv1 cells in normoxia (N) vs. hypoxia (H) in vitro conditions and in control shRNA vs. HIF-1α shRNA-KD under these same conditions. 100,000 cells loaded/lane.

FIG. 34 shows in vivo inhibition of growth of parental control vs. HIF-1α shRNA KD xenografts in untreated castrate mice or mice given a daily oral dose of TasQ [10 mg/kg/d, (n=5/group)].

FIG. 35 shows in vivo growth response of CWR-22Rv1 parental control cells vs. cells expressing the dominant-positive mutant fl-HIF-1α in untreated castrate mice vs. mice with the parental vs. mutant fl-HIF-1α treated daily with TasQ (10 mg/kg/d) starting when the xenografts were 0.05-0.1 cc in size. Insert: IB of flag-tagged HIF-1α protein in parental wt CWR-22Rv1 vs. cells transfected with flag-tagged wt fl-HIF-1α or fl-HIF-1α in which the N-terminal lysine 10, 11, 12, 19, and 21 are mutated to arginine control (i.e. dominant-positive mutant) under normoxia (N) or hypoxia (H) conditions. 100,000 cells loaded/lane.

FIG. 36 shows level of nuclear NCoR1 immunoprecipitated with MEF-2 antibody in CWR-22Rv1 cells under normoxia vs. hypoxia with or without treatment with TasQ normalized to normoxic controls.

FIG. 37 shows sprouting in 3D-fibrin gels with and without HDF-CM by wt vs. HIF-1α KD or HDAC4 KD HUVEC vs. wt cells treated with TasQ. Results are expressed as number of sprouts per HUVEC-coated microcarrier bead.

FIG. 38 shows IB of MEF-2c in HUVEC in 2D vs. 3D-fibrin culture. 100,000 cells loaded/lane.

FIG. 39 shows IB of HIF-1α in wt vs. control shRNA vs. HIF-1α KD HUVEC with or without exposure to cobalt chloride (CoCl₂) to mimic hypoxia. 100,000 cells loaded/lane.

FIG. 40 shows a summary of % PAL AHR binding inhibited by indicated concentration of TasQ vs. ESATA-20.

FIG. 41 shows data when equal aliquots of liver cytosol isolated from hAHR transgenic mice were subjected to competitive ligand binding assays using a fixed saturating dose of photo-affinity ligand (PAL) 2-azido-3-[¹²⁵I]iodo-7,8-dibromodibenzo-p-dioxin and increasing amounts of TasQ or ESATA-20 and photo-cross linked and then cytosol PAGE separated. Left panels: protein stained with Ponceau Red to document equal protein loading and Right panels: autoradiograhy of ¹²⁵I-labeled hAHR at indicated concentration of TasQ vs. ESATA-20.

FIG. 42 shows nuclear HIF-1α protein level under hypoxia in untreated wt LNCaP cells vs. treated with TasQ or ESATA-20 vs. HIF-1α KD or HDAC4 KD cells.

FIG. 43 shows level of nuclear NCoR1 immunoprecipitated with MEF-2 antibody in CWR-22Rv1 cells under normoxia vs. hypoxia with or without treatment with TasQ or ESATA-20 normalized to normoxic controls.

FIG. 44 shows sprouting in 3D-fibrin gels with and without HDF-CM by wt vs. HIF-1α KD or HDAC4 KD HUVEC vs. wt cells treated with TasQ or ESATA-20. Results are expressed as number of sprouts per HUVEC-coated microcarrier bead.

FIG. 45 shows dose-response inhibition of in vivo growth of CWR22-RH human prostate cancer PDXs in castrate male NSG mice over a 3-week treatment period with oral daily TasQ or ESATA-20 initiated when xenografts were ˜200 mm³.

FIG. 46 shows relative expression of mouse Liver Cyp1A1 (mCYP1A1) in animals (n=3) 24 hrs after receiving indicated oral dose of TasQ vs. ESATA-20 normalized to animals receiving only vehicle.

FIG. 47 shows activity of the panel of kinases presented in Supplemental Table 1 in the presence of ESATA-20 (10 μM) analyzed by the Kinase Profiling Service at ThermoFisher Scientific. Data is presented as the mean of 2 replicates+/−SEM. Dashed lines represent +/−15% change in activity.

FIGS. 48A-48D show in vitro growth response to 10 μM of TasQ vs. ESATA20 in BCap-1 (FIG. 48A), LgCap-1 (FIG. 48B), CWR-22Rᵥ1 (FIG. 48C), and LNCaP cell lines (FIG. 48D). In FIG. 48B, such treatment was combined with 3 nM docetaxel.

### DETAILED DESCRIPTION

Preclinical studies have documented that Linomide and its quinolone-3-carboxamide analog, laquinimod, are Aryl Hydrocarbon Receptor (AHR) agonists that induce its target gene effects (i.e., induction of phase I activating enzymes, such as CYP1 A1, throughout the body as well as inducing thymus regression and immune suppression via upregulation of T-reg suppressor cells). This raises the question of whether TasQ is also an AHR agonist and whether this is the mechanism of action for its anti-cancer efficacy. TasQ is also a low nM allosteric inhibitor of HDAC4; its binding prevents HDAC4's ability to epigenetically upregulate stress survival pathways, including those needed for tumor angiogenesis within the compromised tumor microenvironment. This raises the additional question of whether TasQ's anti-cancer mechanism of action requires AHR binding with its associated dose-limiting adverse side effects, or whether this positive effect is AHR-independent due to HDAC4 inhibition.

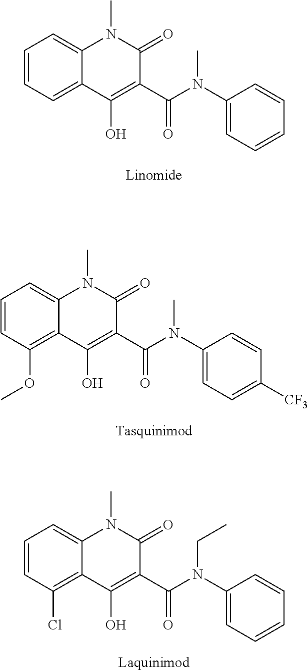 [SMILES: CN(C(=O)c1c(O)c2ccccc2n(C)c1=O)c1ccccc1 ; COc1cccc2c1c(O)c(C(=O)N(C)c1ccc(C(F)(F)F)cc1)c(=O)n2C ; CCN(C(=O)c1c(O)c2c(Cl)cccc2n(C)c1=O)c1ccccc1]

Disclosed herein are studies demonstrating that TasQ is an AHR agonist with an ED₅₀ of 1 μM. This is consistent with the fact that in humans, the maximum tolerated dose (MTD) of TasQ is 1 mg resulting in serum Cmax of ˜0.5 μM, and thus little AHR-dependent dose-limiting host toxicity. However, maximal anti-prostate cancer efficacy requires a serum Cmax of >10 μM, a concentration at which TasQ is a potent AHR agonist, producing host toxicity (i.e. thymus regression and liver CYP1 A1 induction). These combined results are consistent with why the MTD for TasQ is only 1 mg/day and why, at this dose-limiting toxicity, a sub-optimal positive clinical response is produced.

Also disclosed herein are studies demonstrating that lethal in vivo growth of prostate cancers is highly stimulated by HDAC4-dependent transcription of cell stress survival genes, particularly those regulated by HIF1. This involves HDAC4 co-binding NCoR1/HDAC3 and client proteins, like histones and HIF1α. This results in deacetylation of such HDAC4 bound client proteins by the NCoR1/HDAC3 complex. Accordingly, prostate cancer growth characteristically is inhibited by HDAC4 downregulation. While TasQ is an AHR agonist, studies presented herein demonstrate that TasQ's therapeutic efficacy is independent of AHR. Instead, TasQ's anti-cancer mechanism of action involves binding to the “open conformation” of HDAC4, allosterically preventing binding to NCoR1/HDAC3 complex and resulting in the suppression of HIF1 target gene transcription, and thus phenocopying HDAC4 KD.

Accordingly, disclosed herein are third-generation quinolone-3-carboxamide analogs which, in some embodiments, retain potent HDAC4 inhibition while decreasing high-affinity binding to AHR. This may allow higher daily oral doses of the analog to be given to humans, thus increasing anti-prostate cancer therapeutic efficacy.

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents, and other references mentioned herein are incorporated by reference in their entirety. The materials, methods, and examples disclosed herein are illustrative only and not intended to be limiting.

For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Sorrell, Organic Chemistry, 2^{nd} edition, University Science Books, Sausalito, 2006; Smith, March's Advanced Organic Chemistry: Reactions, Mechanism, and Structure, 7^{th} Edition, John Wiley & Sons, Inc., New York, 2013; Larock, Comprehensive Organic Transformations, 3^{rd} Edition, John Wiley & Sons, Inc., New York, 2018; and Carruthers, Some Modern Methods of Organic Synthesis, 3^{rd} Edition, Cambridge University Press, Cambridge, 1987; the entire contents of each of which are incorporated herein by reference.

As used herein, the term “alkyl” means a straight or branched, saturated hydrocarbon chain. Representative examples of alkyl include, but are not limited to, methyl, ethyl, n-propyl, iso-propyl, n-butyl, sec-butyl, iso-butyl, tent-butyl, n-pentyl, isopentyl, neopentyl, n-hexyl, 3-methylhexyl, 2,2-dimethylpentyl, 2,3-dimethylpentyl, 4,4-dimethylpentan-2-yl, n-heptyl, n-octyl, n-nonyl, n-decyl, undecyl, dodecyl, tridecyl, tetradecyl, pentadecyl, hexadecyl, heptadecyl, octadecyl, nonadecyl and icosyl.

As used herein, the term “alkoxy” refers to an alkyl group, as defined herein, appended to the parent molecular moiety through an oxygen atom. Representative examples of alkoxy include, but are not limited to, methoxy, ethoxy, n-propoxy, isopropoxy, n-butoxy, and tert-butoxy.

As used herein, the term “amino” refers to an —NH₂ group. As used herein, the term “alkylamino” refers to a group —NHR, wherein R is an alkyl group as defined herein. As used herein, the term “dialkylamino” refers to a group —NR₂, wherein each R is independently an alkyl group as defined herein.

As used herein, “aminoalkyl” refers to an alkyl group, as defined herein, in which at least one hydrogen atom (e.g., one hydrogen atom) is replaced with an amino group.

As used herein, the term “aryl” refers to an aromatic carbocyclic ring system having a single ring (monocyclic) or multiple rings (bicyclic or tricyclic) including fused ring systems, and zero heteroatoms. As used herein, aryl contains 6-20 carbon atoms (C₆-C₂₀ aryl), 6 to 14 ring carbon atoms (C₆-C₁₄ aryl), 6 to 12 ring carbon atoms (C₆-C₁₂ aryl), or 6 to 10 ring carbon atoms (C₆-C₁₀ aryl). Representative examples of aryl groups include, but are not limited to, phenyl, naphthyl, anthracenyl, and phenanthrenyl.

As used herein, the term “arylalkyl” refers to an means an alkyl group, as defined herein, in which at least one hydrogen atom is replaced with an aryl group, as defined herein. Representative examples of arylalkyl include, but are not limited to, benzyl and phenethyl.

As used herein, the term “arylalkyloxy” refers to an means an arylalkyl group, as defined herein, appended to the parent molecular moiety through an oxygen atom.

As used herein, the term “cycloalkyl” refers to a saturated carbocyclic ring system containing three to ten carbon atoms and zero heteroatoms. The cycloalkyl may be monocyclic, bicyclic, bridged, fused, or spirocyclic. Representative examples of cycloalkyl include, but are not limited to, cyclopropyl, cyclobutyl, cyclopentyl, cyclohexyl, cycloheptyl, cyclooctyl, cyclononyl, cyclodecyl, adamantyl, bicyclo[2.2.1]heptanyl, bicyclo[3.2.1]octanyl, and bicyclo[5.2.0]nonanyl.

As used herein, the term “halogen” or “halo” means F, Cl, Br, or 1.

As used herein, the term “haloalkyl” means an alkyl group, as defined herein, in which at least one hydrogen atom (e.g., one, two, three, four, five, six, seven or eight hydrogen atoms) is replaced with a halogen. In some embodiments, each hydrogen atom of the alkyl group is replaced with a halogen. Representative examples of haloalkyl include, but are not limited to, fluoromethyl, difluoromethyl, trifluoromethyl, 2-fluoroethyl, 2,2,2-trifluoroethyl, and 3,3,3-trifluoropropyl.

As used herein, the term “hydroxyalkyl” refers to an alkyl group, as defined herein, in which at least one hydrogen atom (e.g., one hydrogen atom) is replaced with a hydroxy group.

As used herein, the term “subject” broadly refers to any animal, including but not limited to, human and non-human animals (e.g., dogs, cats, cows, horses, sheep, poultry, etc.). As used herein, the term “patient” typically refers to a subject that is being treated for a disease or condition.

As used herein, the terms “treatment,” “treat,” and “treating” refer to reversing, alleviating, delaying the onset of, or inhibiting the progress of a disease or condition, or one or more signs or symptoms thereof. In some embodiments, “treatment,” “treat,” and “treating” require that signs or symptoms of the disease disorder or condition have developed or have been observed. In other embodiments, treatment may be administered in the absence of signs or symptoms of the disease or condition. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 2. Compounds

The present disclosure includes compounds of formula (I):

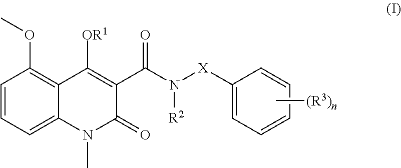

or a pharmaceutically acceptable salt thereof, wherein:
R¹ is selected from hydrogen and C₁-C₄ alkyl, C₁-C₄ hydroxyalkyl, and C₁-C₄ aminoalkyl, wherein the amino of the C₁-C₄ aminoalkyl is optionally protected by a protecting group;
R² is selected from hydrogen, C₃-C₁₀ alkyl, C₃-C₆ cycloalkyl, and arylalkyl, wherein the alkyl, cycloalkyl, and arylalkyl are each optionally substituted with 1, 2, or 3 substituents independently selected from halo, C₁-C₄ haloalkyl, hydroxy, C₁-C₄ alkoxy, and arylalkyloxy;
n is 0, 1, 2, or 3;
each R³ is independently selected from C₁-C₄ haloalkyl, halo, C₁-C₄ alkyl, and C₁-C₄ alkoxy; and
X is a bond or —N═CH—, wherein if X is a bond, R² is not hydrogen.

In some embodiments, R¹ is selected from hydrogen, methyl, ethyl, and 2-aminoethyl, wherein the amino of the 2-aminoethyl is optionally protected by a tert-butyloxycarbonyl group. In some embodiments, R¹ is hydrogen.

In some embodiments, X is a bond, and R² is selected from C₃-C₁₀ alkyl, C₃-C₆ cycloalkyl, and arylalkyl, wherein the alkyl, cycloalkyl, and arylalkyl are each optionally substituted with 1, 2, or 3 substituents independently selected from halo, C₁-C₄ haloalkyl, hydroxy, C₁-C₄ alkoxy, and arylalkyloxy. In some embodiments, X is a bond, and R² is selected from isopropyl, n-heptyl, cyclohexyl, benzyl, and ethyl substituted with one benzyloxy group. In some embodiments, X is a bond, and R² is selected from C₄-C₆ alkyl and C₃-C₆ cycloalkyl.

In some embodiments, X is —N═CH— and R² is hydrogen.

In some embodiments, n is 0.

In some embodiments, n is 1. In some embodiments, R³ is substituted at the para position of the phenyl group. In some embodiments, R3 is C₁-C₄ haloalkyl. In some embodiments, R³ is trifluoromethyl.

In some embodiments, the compound is a compound of formula (Ia):

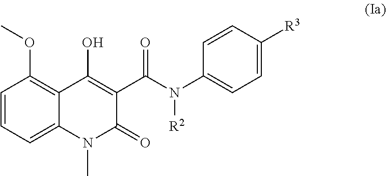

or a pharmaceutically acceptable salt thereof, wherein:
R² is selected from C₄-C₆, alkyl and C₃-C₆ cycloalkyl; and
R³ is independently selected from C₁-C₄ haloalkyl, halo, C₁-C₄ alkyl, and C₁-C₄ alkoxy.

In some embodiments, R² is C₃-C₆ cycloalkyl, and R³ is C₁-C₄ haloalkyl. In some embodiments, R² is cyclohexyl. In some embodiments, R³ is trifluoromethyl.

In some embodiments, the compound is selected from:

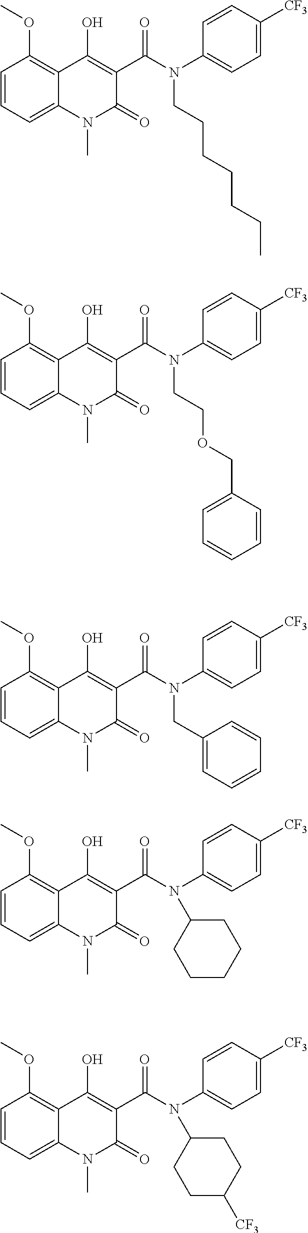

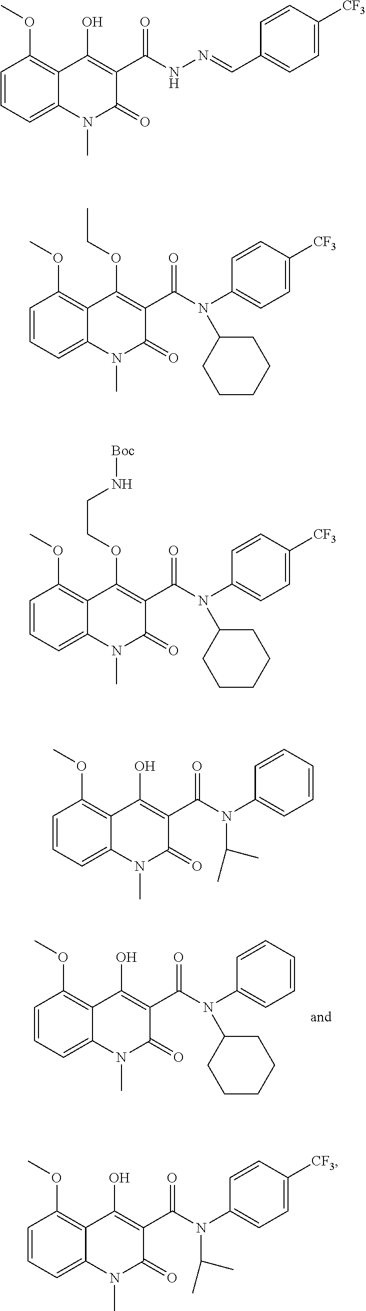 [SMILES: COc1cccc2c1c(O)c(C(=O)N/N=C/c1ccc(C(F)(F)F)cc1)c(=O)n2C ; CCOc1c(C(=O)N(c2ccc(C(F)(F)F)cc2)C2CCCCC2)c(=O)n(C)c2cccc(OC)c12 ; COc1cccc2c1c(OCCNC(=O)OC(C)(C)C)c(C(=O)N(c1ccc(C(F)(F)F)cc1)C1CCCCC1)c(=O)n2C ; COc1cccc2c1c(O)c(C(=O)N(c1ccccc1)C(C)C)c(=O)n2C ; COc1cccc2c1c(O)c(C(=O)N(c1ccccc1)C1CCCCC1)c(=O)n2C ; COc1cccc2c1c(O)c(C(=O)N(c1ccc(C(F)(F)F)cc1)C(C)C)c(=O)n2C]

or a pharmaceutically acceptable salt thereof.

In some embodiments, the compound is:

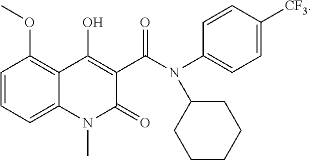 [SMILES: COc1cccc2c1c(O)c(C(=O)N(c1ccc(C(F)(F)F)cc1)C1CCCCC1)c(=O)n2C]

The compounds can be in the form of a salt. In some embodiments, a neutral form of the compound may be regenerated by contacting the salt with a base or acid and isolating the parent compound in a conventional manner. The parent form of the compound differs from the various salt forms in certain physical properties, such as solubility in polar solvents, but otherwise the salts are equivalent to the parent form of the compound for the purposes of this disclosure.

In particular, if the compound is anionic or has a functional group that may be anionic, then a salt may be formed with one or more suitable cations. Examples of suitable inorganic cations include, but are not limited to, alkali metal cations such as Li⁺, Na⁺, and K⁺, alkaline earth cations such as Ca²⁺ and Mg²⁺, and other cations. Sodium salts may be particularly suitable. Examples of suitable organic cations include, but are not limited to, ammonium ion (i.e., NH₄⁺) and substituted ammonium ions (e.g., NH₃R₁⁺, NH₂R₂⁺, NHR₃⁺, and NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids such as lysine and arginine. In some embodiments, the compound is a sodium salt.

If the compound is cationic or has a functional group that may be cationic (e.g., an amino group that can be protonated), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous. Examples of suitable organic anions include, but are not limited to, those derived from the following organic acids; 2-acetyoxybenzoic, acetic, ascorbic, aspartic, benzoic, camphorsulfonic, cinnamic, citric, edetic, ethanedisulfonic, ethanesulfonic, fumaric, glucoheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, tetrafluoroboric, toluenesulfonic, trifluoromethanesulfonic, and valeric. In some embodiments, the compound is a halide salt, such as a chloro, bromo, or iodo salt. In some embodiments, the compound is a tetrafluoroborate or trifluoromethanesulfonate salt.

The present disclosure also includes isotopically-labeled compounds (e.g., an isotopically-labeled compound of formula (I)), which are identical to those recited in formula (I), but for the fact that one or more atoms are replaced by an atom having an atomic mass or mass number different from the atomic mass or mass number usually found in nature. Examples of isotopes suitable for inclusion in the compounds of the disclosure are hydrogen, carbon, nitrogen, oxygen, phosphorus, sulfur, fluorine, and chlorine, such as, but not limited to ²H, ³H, ¹³C, ¹⁴C, ¹⁵N, ¹⁸O, ³¹P, ³⁵S, ¹⁸F, and ³⁶Cl, respectively. Substitution with heavier isotopes such as deuterium, i.e. 2H, can afford certain therapeutic advantages resulting from greater metabolic stability, for example increased in vivo half-life or reduced dosage requirements and, hence, may be preferred in some circumstances. The compound may incorporate positron-emitting isotopes for medical imaging and positron-emitting tomography (PET) studies for determining the distribution of receptors. Suitable positron-emitting isotopes that can be incorporated in compounds of formula (I) are ¹¹C, ¹³N, ¹⁵O, and ¹⁸F. Isotopically-labeled compounds of formula (I) can generally be prepared by conventional techniques known to those skilled in the art or by processes analogous to those described herein using an appropriate isotopically-labeled reagent in place of a non-isotopically-labeled reagent.

Compounds of the disclosure can also be functionalized with one or more groups such as targeting ligands, biopolymers, polyethylene glycol, and the like. A group such as a targeting ligand can be attached, for example, via a linker. In some embodiments, the linker is a cleavable linker. In some embodiments, the targeting ligand is a ligand that targets prostate-specific antigen (PSA), prostate-specific membrane antigen (PSMA), folate receptor, fibroblast activation protein (FAP), or hexokinase 2 (HK2).

The compounds can be prepared by a variety of methods, including those illustrated in the Examples. The compounds and intermediates herein may be isolated and purified by methods well-known to those skilled in the art of organic synthesis. Examples of conventional methods for isolating and purifying compounds can include, but are not limited to, chromatography on solid supports such as silica gel, alumina, or silica derivatized with alkylsilane groups, by recrystallization at high or low temperature with an optional pretreatment with activated carbon, thin-layer chromatography, distillation at various pressures, sublimation under vacuum, and trituration as described for instance in “Vogel's Textbook of Practical Organic Chemistry,” 5th edition (1989), by Furniss, Hannaford, Smith, and Tatchell, pub. Longman Scientific & Technical, Essex CM20 2JE, England.

Reaction conditions and reaction times for each individual step can vary depending on the particular reactants employed and substituents present in the reactants used. Reactions can be worked up in the conventional manner, e.g., by eliminating the solvent from the residue and further purified according to methodologies generally known in the art such as, but not limited to, crystallization, distillation, extraction, trituration, and chromatography. Unless otherwise described, the starting materials and reagents are either commercially available or can be prepared by one skilled in the art from commercially available materials using methods described in the chemical literature. Starting materials, if not commercially available, can be prepared by procedures selected from standard organic chemical techniques, techniques that are analogous to the synthesis of known, structurally similar compounds, or techniques that are analogous to the above described schemes or the procedures described in the synthetic examples section.

Routine experimentations, including appropriate manipulation of the reaction conditions, reagents and sequence of the synthetic route, protection of any chemical functionality that cannot be compatible with the reaction conditions, and deprotection at a suitable point in the reaction sequence of the method are included in the scope of the invention. Suitable protecting groups, and the methods for protecting and deprotecting different substituents using such suitable protecting groups, are well known to those skilled in the art; examples of which can be found in the treatise by PGM Wuts entitled “Greene's Protective Groups in Organic Synthesis” (5th ed.), John Wiley & Sons, Inc. (2014), which is incorporated herein by reference in its entirety. Synthesis of the compounds of the invention can be accomplished by methods analogous to those described in the synthetic schemes described hereinabove and in specific examples.

When an optically active form of a disclosed compound is required, it can be obtained by carrying out one of the procedures described herein using an optically active starting material (prepared, for example, by asymmetric induction of a suitable reaction step) or by resolution of a mixture of the stereoisomers of the compound or intermediates using a standard procedure (such as chromatographic separation, recrystallization or enzymatic resolution).

Similarly, when a pure geometric isomer of a compound is required, it can be obtained by carrying out one of the above procedures using a pure geometric isomer as a starting material or by resolution of a mixture of the geometric isomers of the compound or intermediates using a standard procedure such as chromatographic separation.

The synthetic schemes and specific examples as described are illustrative and are not to be read as limiting the scope of the invention as it is defined in the claims. All alternatives, modifications, and equivalents of the synthetic methods and specific examples are included within the scope of the claims.

### 3. Pharmaceutical Compositions

The disclosed compounds may be incorporated into pharmaceutical compositions suitable for administration to a subject (such as a patient, which may be a human or non-human). Accordingly, in some embodiments, the present disclosure provides a pharmaceutical composition comprising a compound disclosed herein (i.e. a compound of formula (I), or a pharmaceutically acceptable salt thereof, and a pharmaceutically acceptable carrier.

The pharmaceutical compositions may include a “therapeutically effective amount” or a “prophylactically effective amount” of the agent. A “therapeutically effective amount” refers to an amount effective, at dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects of a compound of the disclosure (e.g., a compound of formula (I)) are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

For example, a therapeutically effective amount of a compound of formula (I), may be about 1 mg/kg to about 1000 mg/kg, about 5 mg/kg to about 950 mg/kg, about 10 mg/kg to about 900 mg/kg, about 15 mg/kg to about 850 mg/kg, about 20 mg/kg to about 800 mg/kg, about 25 mg/kg to about 750 mg/kg, about 30 mg/kg to about 700 mg/kg, about 35 mg/kg to about 650 mg/kg, about 40 mg/kg to about 600 mg/kg, about 45 mg/kg to about 550 mg/kg, about 50 mg/kg to about 500 mg/kg, about 55 mg/kg to about 450 mg/kg, about 60 mg/kg to about 400 mg/kg, about 65 mg/kg to about 350 mg/kg, about 70 mg/kg to about 300 mg/kg, about 75 mg/kg to about 250 mg/kg, about 80 mg/kg to about 200 mg/kg, about 85 mg/kg to about 150 mg/kg, and about 90 mg/kg to about 100 mg/kg.

The pharmaceutical compositions include pharmaceutically acceptable carriers. The term “pharmaceutically acceptable carrier,” as used herein, means a nontoxic, inert solid, semi-solid or liquid filler, diluent, encapsulating material auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin: talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Thus, the compounds and their physiologically acceptable salts may be formulated for administration by, for example, solid dosing, eye drop, in a topical oil-based formulation, injection, inhalation (either through the mouth or the nose), implants, or oral, buccal, parenteral, or rectal administration. Techniques and formulations may generally be found in “Remington's Pharmaceutical Sciences” (Meade Publishing Co., Easton, Pa.). Therapeutic compositions must typically be sterile and stable under the conditions of manufacture and storage.

The route by which the disclosed compounds are administered and the form of the composition will dictate the type of carrier to be used. The composition may be in a variety of forms, suitable, for example, for systemic administration (e.g., oral, rectal, nasal, sublingual, buccal, implants, or parenteral) or topical administration (e.g., dermal, pulmonary, nasal, aural, ocular, liposome delivery systems, or iontophoresis).

Carriers for systemic administration typically include at least one of diluents, lubricants, binders, disintegrants, colorants, flavors, sweeteners, antioxidants, preservatives, glidants, solvents, suspending agents, wetting agents, surfactants, combinations thereof, and others. All carriers are optional in the compositions.

Suitable diluents include sugars such as glucose, lactose, dextrose, and sucrose; diols such as propylene glycol; calcium carbonate; sodium carbonate; sugar alcohols, such as glycerin; mannitol; and sorbitol. The amount of diluent(s) in a systemic or topical composition is typically about 50 to about 90%.

Suitable lubricants include silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of theobroma. The amount of lubricant(s) in a systemic or topical composition is typically about 5 to about 10%.

Suitable binders include polyvinyl pyrrolidone; magnesium aluminum silicate; starches such as corn starch and potato starch; gelatin: tragacanth; and cellulose and its derivatives, such as sodium carboxymethylcellulose, ethyl cellulose, methylcellulose, microcrystalline cellulose, and sodium carboxymethylcellulose. The amount of binder(s) in a systemic composition is typically about 5 to about 50%.

Suitable disintegrants include agar, alginic acid and the sodium salt thereof, effervescent mixtures, croscarmellose, crospovidone, sodium carboxymethyl starch, sodium starch glycolate, clays, and ion exchange resins. The amount of disintegrant(s) in a systemic or topical composition is typically about 0.1 to about 10%.

Suitable colorants include a colorant such as an FD&C dye. When used, the amount of colorant in a systemic or topical composition is typically about 0.005 to about 0.1%.

Suitable flavors include menthol, peppermint, and fruit flavors. The amount of flavor(s), when used, in a systemic or topical composition is typically about 0.1 to about 1.0%.

Suitable sweeteners include aspartame and saccharin. The amount of sweetener(s) in a systemic or topical composition is typically about 0.001 to about 1%.

Suitable antioxidants include butylated hydroxyanisole (“BHA”), butylated hydroxytoluene (“BHT”), and vitamin E. The amount of antioxidant(s) in a systemic or topical composition is typically about 0.1 to about 5%.

Suitable preservatives include benzalkonium chloride, methyl paraben and sodium benzoate. The amount of preservative(s) in a systemic or topical composition is typically about 0.01 to about 5%.

Suitable glidants include silicon dioxide. The amount of glidant(s) in a systemic or topical composition is typically about 1 to about 5%.

Suitable solvents include water, isotonic saline, ethyl oleate, glycerine, hydroxylated castor oils, alcohols such as ethanol, and phosphate buffer solutions. The amount of solvent(s) in a systemic or topical composition is typically from about 0 to about 100%.

Suitable suspending agents include AVICEL RC-591 (from FMC Corporation of Philadelphia, PA) and sodium alginate. The amount of suspending agent(s) in a systemic or topical composition is typically about 1 to about 8%.

Suitable surfactants include lecithin, Polysorbate 80, and sodium lauryl sulfate, and the TWEENS from Atlas Powder Company of Wilmington, Delaware. Suitable surfactants include those disclosed in the C.T.F.A. Cosmetic Ingredient Handbook, 1992, pp. 587-592; Remington's Pharmaceutical Sciences, 15th Ed. 1975, pp. 335-337; and McCutcheon's Volume 1, Emulsifiers & Detergents, 1994, North American Edition, pp. 236-239. The amount of surfactant(s) in the systemic or topical composition is typically about 0.1% to about 5%.

Although the amounts of components in the systemic compositions may vary depending on the type of systemic composition prepared, in general, systemic compositions include 0.01% to 50% of an active compound (e.g., a compound of formula (I)) and 50% to 99.99% of one or more carriers. Compositions for parenteral administration typically include 0.1% to 10% of actives and 90% to 99.9% of a carrier including a diluent and a solvent.

Compositions for oral administration can have various dosage forms. For example, solid forms include tablets, capsules, granules, and bulk powders. These oral dosage forms include a safe and effective amount, usually at least about 5%, and more particularly from about 25% to about 50% of actives. The oral dosage compositions include about 50% to about 95% of carriers, and more particularly, from about 50% to about 75%.

Tablets can be compressed, tablet triturates, enteric-coated, sugar-coated, film-coated, or multiple-compressed. Tablets typically include an active component, and a carrier comprising ingredients selected from diluents, lubricants, binders, disintegrants, colorants, flavors, sweeteners, glidants, and combinations thereof. Specific diluents include calcium carbonate, sodium carbonate, mannitol, lactose and cellulose. Specific binders include starch, gelatin, and sucrose. Specific disintegrants include alginic acid and croscarmellose. Specific lubricants include magnesium stearate, stearic acid, and talc. Specific colorants are the FD&C dyes, which can be added for appearance. Chewable tablets preferably contain sweeteners such as aspartame and saccharin, or flavors such as menthol, peppermint, fruit flavors, or a combination thereof.

Capsules (including implants, time release and sustained release formulations) typically include an active compound (e.g., a compound of formula (I)), and a carrier including one or more diluents disclosed above in a capsule comprising gelatin. Granules typically comprise a disclosed compound, and preferably glidants such as silicon dioxide to improve flow characteristics. Implants can be of the biodegradable or the non-biodegradable type.

The selection of ingredients in the carrier for oral compositions depends on secondary considerations like taste, cost, and shelf stability, which are not critical for the purposes of this disclosure.

Solid compositions may be coated by conventional methods, typically with pH or time-dependent coatings, such that a disclosed compound is released in the gastrointestinal tract in the vicinity of the desired application, or at various points and times to extend the desired action. The coatings typically include one or more components selected from the group consisting of cellulose acetate phthalate, polyvinyl acetate phthalate, hydroxypropyl methyl cellulose phthalate, ethyl cellulose, EUDRAGIT® coatings (available from Evonik Industries of Essen, Germany), waxes and shellac.

Compositions for oral administration can have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically include a disclosed compound and a carrier, namely, a carrier selected from diluents, colorants, flavors, sweeteners, preservatives, solvents, suspending agents, and surfactants. Peroral liquid compositions preferably include one or more ingredients selected from colorants, flavors, and sweeteners.

Other compositions useful for attaining systemic delivery of the subject compounds include sublingual, buccal and nasal dosage forms. Such compositions typically include one or more of soluble filler substances such as diluents including sucrose, sorbitol and mannitol; and binders such as acacia, microcrystalline cellulose, carboxymethyl cellulose, and hydroxypropyl methylcellulose. Such compositions may further include lubricants, colorants, flavors, sweeteners, antioxidants, and glidants.

The disclosed compounds can be topically administered. Topical compositions that can be applied locally to the skin may be in any form including solids, solutions, oils, creams, ointments, gels, lotions, shampoos, leave-on and rinse-out hair conditioners, milks, cleansers, moisturizers, sprays, skin patches, and the like. Topical compositions include: a disclosed compound (e.g., a compound of formula (I)), and a carrier. The carrier of the topical composition preferably aids penetration of the compounds into the skin. The carrier may further include one or more optional components.

The amount of the carrier employed in conjunction with a disclosed compound is sufficient to provide a practical quantity of composition for administration per unit dose of the compound. Techniques and compositions for making dosage forms useful in the methods of this disclosure are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2nd Ed., (1976).

A carrier may include a single ingredient or a combination of two or more ingredients. In the topical compositions, the carrier includes a topical carrier. Suitable topical carriers include one or more ingredients selected from phosphate buffered saline, isotonic water, deionized water, monofunctional alcohols, symmetrical alcohols, aloe vera gel, allantoin, glycerin, vitamin A and E oils, mineral oil, propylene glycol, PPG-2 myristyl propionate, dimethyl isosorbide, castor oil, combinations thereof, and the like. More particularly, carriers for skin applications include propylene glycol, dimethyl isosorbide, and water, and even more particularly, phosphate buffered saline, isotonic water, deionized water, monofunctional alcohols, and symmetrical alcohols.

The carrier of a topical composition may further include one or more ingredients selected from emollients, propellants, solvents, humectants, thickeners, powders, fragrances, pigments, and preservatives, all of which are optional.

Suitable emollients include stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. Specific emollients for skin include stearyl alcohol and polydimethylsiloxane. The amount of emollient(s) in a skin-based topical composition is typically about 5% to about 95%.

Suitable propellants include propane, butane, isobutane, dimethyl ether, carbon dioxide, nitrous oxide, and combinations thereof. The amount of propellant(s) in a topical composition is typically about 0% to about 95%.

Suitable solvents include water, ethyl alcohol, methylene chloride, isopropanol, castor oil, ethylene glycol monoethyl ether, diethylene glycol monobutyl ether, diethylene glycol monoethyl ether, dimethylsulfoxide, dimethyl formamide, tetrahydrofuran, and combinations thereof. Specific solvents include ethyl alcohol and homotopic alcohols. The amount of solvent(s) in a topical composition is typically about 0% to about 95%.

Suitable humectants include glycerin, sorbitol, sodium 2-pyrrolidone-5-carboxylate, soluble collagen, dibutyl phthalate, gelatin, and combinations thereof. Specific humectants include glycerin. The amount of humectant(s) in a topical composition is typically 0% to 95%.

The amount of thickener(s) in a topical composition is typically about 0% to about 95%.

Suitable powders include beta-cyclodextrins, hydroxypropyl cyclodextrins, chalk, talc, fullers earth, kaolin, starch, gums, colloidal silicon dioxide, sodium polyacrylate, tetra alkyl ammonium smectites, trialkyl aryl ammonium smectites, chemically-modified magnesium aluminum silicate, organically-modified montmorillonite clay, hydrated aluminum silicate, fumed silica, carboxyvinyl polymer, sodium carboxymethyl cellulose, ethylene glycol monostearate, and combinations thereof. The amount of powder(s) in a topical composition is typically 0% to 95%.

The amount of fragrance in a topical composition is typically about 0% to about 0.5%, particularly, about 0.001% to about 0.1%.

Suitable pH adjusting additives include HCl or NaOH in amounts sufficient to adjust the pH of a topical pharmaceutical composition.

### 4. Methods of Treatment

Embodiments of the present disclosure include methods of treating cancer in a subject in need thereof, comprising administering to the subject a therapeutically effective amount of a compound described herein (e.g., a compound of formula (I), or a pharmaceutically acceptable salt thereof).

In some embodiments, the disclosure provides a method of treating prostate cancer in a subject in need thereof, comprising administering to the subject a therapeutically effective amount of a compound described herein (e.g., a compound of formula (I) or a pharmaceutically acceptable salt thereof), or a pharmaceutical composition described herein (e.g., a pharmaceutical composition comprising a compound of formula (I) or a pharmaceutically acceptable salt thereof). In some embodiments, the prostate cancer is hormone-dependent prostate cancer. In some embodiments, the prostate cancer is hormone-independent prostate cancer. In some embodiments, the prostate cancer is castration-resistant prostate cancer. In some embodiments, the cancer is metastatic castrate-resistant prostate cancer.

In the methods described herein, a compound or pharmaceutical composition may be administered to the subject by any convenient route of administration, whether systemically/peripherally or at the site of desired action, including but not limited to, oral (e.g. by ingestion); topical (including e.g. transdermal, intranasal, ocular, buccal, and sublingual); pulmonary (e.g. by inhalation or insufflation therapy using, e.g. an aerosol, e.g. through mouth or nose); rectal; vaginal; parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; or by implant of a depot, for example, subcutaneously or intramuscularly. In some embodiments, the administration comprises oral administration. Additional modes of administration may include adding the compound and/or a composition comprising the compound to a food or beverage, including a water supply for an animal, to supply the compound as part of the animal's diet.

It will be appreciated that appropriate dosages of the compounds, and compositions comprising the compounds, can vary from patient to patient. Determining the optimal dosage will generally involve the balancing of the level of therapeutic benefit against any risk or deleterious side effects of the treatments of the present disclosure. The selected dosage level will depend on a variety of factors including, but not limited to, the activity of the particular compound, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations at the site of action which achieve the desired effect without causing substantial harmful or deleterious side-effects.

Administration in vivo can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician. In general, a suitable dose of the compound is in the range of about 100 μg to about 250 mg per kilogram body weight of the subject per day.

The compound or composition may be administered once, on a continuous basis (e.g. by an intravenous drip), or on a periodic/intermittent basis, including about once per hour, about once per two hours, about once per four hours, about once per eight hours, about once per twelve hours, about once per day, about once per two days, about once per three days, about twice per week, about once per week, and about once per month. The composition may be administered until a desired reduction of symptoms is achieved.

A compound described herein may be used in combination with other known therapies. Administered “in combination,” as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

A compound or composition described herein and the at least one additional therapeutic agent can be administered simultaneously, in the same or in separate compositions, or sequentially. For sequential administration, the compound described herein can be administered first, and the additional agent can be administered subsequently, or the order of administration can be reversed.

In some embodiments, a compound described herein is administered in combination with other therapeutic treatment modalities, including surgery, radiation, cryotherapy, cryosurgery, and/or thermotherapy. Such combination therapies may advantageously utilize lower dosages of the administered agent and/or other chemotherapeutic agent, thus avoiding possible toxicities or complications associated with the various therapies. The phrase “radiation” includes, but is not limited to, external-beam therapy which involves three dimensional, conformal radiation therapy where the field of radiation is designed to conform to the volume of tissue treated; interstitial-radiation therapy where seeds of radioactive compounds are implanted using ultrasound guidance; and a combination of external-beam therapy and interstitial-radiation therapy.

In some embodiments, the compound described herein is administered with at least one additional therapeutic agent, such as a chemotherapeutic agent. In certain embodiments, the compound described herein is administered in combination with one or more additional chemotherapeutic agents. The chemotherapeutic agent may be a chemotherapeutic agent identified on the “A to Z List of Cancer Drugs” published by the National Cancer Institute. In some embodiments, the chemotherapeutic agent is selected from abiraterone, apalutamide, bicalutamide, cabazitaxel, capecitabine, cyclophosphamide, darolutamide, degarelix, docetaxel, dutasteride, enzalutamide, estradiol, estramustine, finasteride, flutamide, goserelin, histrelin, leuprolide, mitoxantrone, nilutamide, olaparib, radium-223, rucaparib, sipuleucel-T, and triptorelin. In some embodiments, the chemotherapeutic agent is a taxane, such as such as docetaxel or cabazitaxel.

### 5. Examples

It will be readily apparent to those skilled in the art that other suitable modifications and adaptations of the methods of the present disclosure described herein are readily applicable and appreciable and may be made using suitable equivalents without departing from the scope of the present disclosure or the aspects and embodiments disclosed herein. The disclosure will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

Reagents: Linomide, Tasquinimod [TasQ], Compound ESATA4 and Compound ESATA5 (as shown below) (Active Biotech Research AB); Trichostatin-A (Sigma-Aldrich); trifluoromethyl-acetylysine-7-amino-methylcoumarin (aka Boc-Lys(Tfa)-AMC) [i.e., HDAC4 specific substrate; Bachem Inc.]; HDAC4: rabbit polyclonal Ab (Active Motif, Cat #40969); Flag: clone M2: mouse monoclonal Ab (Sigma-Aldrich, Cat #F3165); HIF-1α(H-206): rabbit polyclonal Ab [Santa Cruz, Cat #sc-10790 (IP)]; HIF-1α: mouse monoclonal Ab [BD Transduction Laboratories, Cat #610958 (LB)]; HIF-1α(H-206): rabbit polyclonal [Bethyl Laboratories, Cat #A300-286A (1B)]; N-CoR: rabbit polyclonal Ab (Bethyl Laboratories, Cat #A301-146A). Full-length (fl) [A.A. 1-1082] recombinant human histone deacetylase 4 (rhHDAC4) protein as either: wild-type or C669/H675-double mutant, or R681A/R798A double mutant protein with or without an N-terminal GST-tag was produced in HEK293-T cells based upon a pcDNA vector containing human N-terminal flag-tag fl wild-type histone deacetylase 4 (HDAC4) obtained from Addgene (Cat #13821) as described previously (Isaacs et al. Cancer Res. 2013, 73:1386-1399). N-terminal GST-tagged truncated recombinant HDAC4 protein (A.A. 614-1084) was obtained from Abcam, Cambridge, UK (cat number ab104029). Recombinant fl-human nuclear receptor co-repressor-1 (NCoR1) protein (N-terminal FLAG-tagged; AA 1-2440) was purchased from Abcam, Cambridge, UK (cat. no. ab82239).

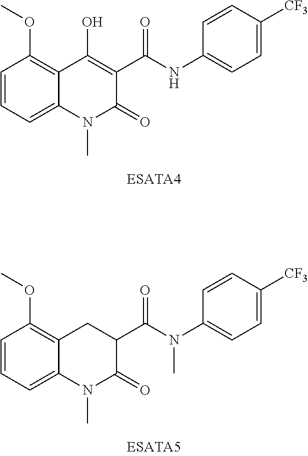 [SMILES: COc1cccc2c1c(O)c(C(=O)Nc1ccc(C(F)(F)F)cc1)c(=O)n2C ; COc1cccc2c1CC(C(=O)N(C)c1ccc(C(F)(F)F)cc1)C(=O)N2C]

Abbreviations used in the Examples include the following: DCM is dichloromethane; DIAD is diisopropyl azodicarboxylate; DIPEA is N,N-diisopropylethylamine, DMF is N,N-dimethylformamide; DMSO is dimethyl sulfoxide; HATU is 1-[bis(dimethylamino)methylene]-1H-1,2,3-triazolo[4,5-b]pyridinium 3-oxide hexafluorophosphate; HPLC is high performance liquid chromatography; NMR is nuclear magnetic resonance; PMHS is polymethylhydrosiloxane; and THF is tetrahydrofuran.

### Example 1

### Compound Syntheses

All solvents and reagents used were bought from commercial sources and used without further purification. The ¹H and ¹³C NMR spectra were obtained on a Bruker Avance III 500 MHz NMR spectrometer at 500 MHz and 125 MHz, respectively in deuterated chloroform (CDCl₃), deuterated methanol (CD₃OD) or deuterated dimethylsulfoxide (C₂D₆OS). MALDI-Mass spectra were obtained on Voyager DE-STR MALDI-TOF. Analytical thin-layer chromatography was performed using 0.25 mm precoated silica gel 60 F254 plates (Anal-tech Uniplates). Flash column chromatography was performed using silica gel 60 (200-400 mesh, Sorbent Technologies). As needed, further purification of synthetic compounds, including all tasquinimod analogs was done with preparative HPLC using Waters Delta 600 Controller equip with a variable wavelength UV-VIS detector. Purity of the compounds was determined using reverse phase-HPLC.

### Synthesis of Intermediate Compound ESATA2

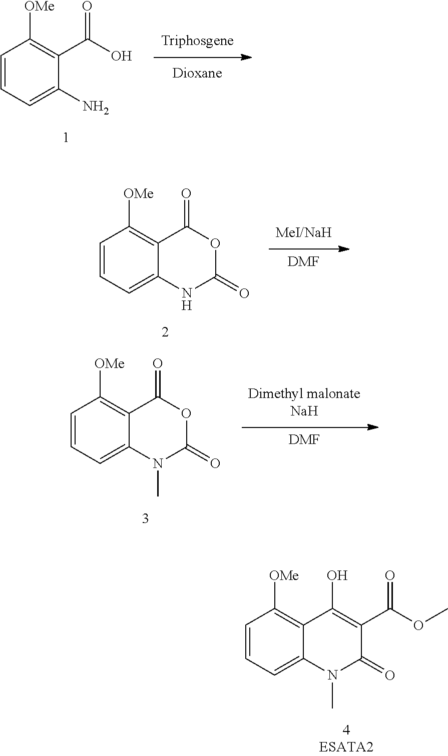

Compound ESATA2 was synthesized as previously reported (J. Med. Chem. 2004; 47: 2075) with some modifications. 1,4-dioxane was placed in a round bottomed flasked in an ice bath. To this was added 1.5 molar equivalents of triphosgene. Then 1 molar equiv of a 2-amino-6-methoxybenzoic acid slurry in DCM was added to the triphosgene solution gradually. The ice bath was then removed and the slurry (light creamy brown solution) was left to stir at room temperature for 2 hours. Solvent was then evaporated completely and the residue was triturated in diethyl ether (because triphosgene is soluble in ether). It was then filtered and washed with diethyl ether and then air dried to give Compound 2 in 99% yield. It was analyzed and characterized by ¹H NMR and ¹³C NMR.

Compound 2 was dissolved in DMF. The solution was placed in an ice bath and cooled to 0° C. Then 1.5 molar equivalents of NaH in 60% mineral oil were added gradually. Then methyl iodide was added dropwise and rinsed down with DMF. The ice bath was removed and the heterogenous solution (slurry) obtained was stirred at room temperature for 3 hours. Solvent and all volatiles were then evaporated completely under reduced pressure to produce Compound 3.

To Compound 3 was added DMF and 2.0 molar equivalents of NaH portionwise. This was followed by addition of 2 molar equivalents of dimethyl malonate dropwise. The mixture was then placed in a oil bath, heated to 130° C. and stirred at this temperature with a reflux condenser connected for 1 hour. The mixture was then cooled to room temperature, and 400 mL of 1.0 M HCl was added. The slurry formed was filtered and then left to dry on suction overnight to give Compound 4 in 93.7% yield. It was analyzed and characterized with ¹H NMR, ¹³C NMR, and HPLC. Its purity was 98% as measured by HPLC.

### Synthesis of Amines

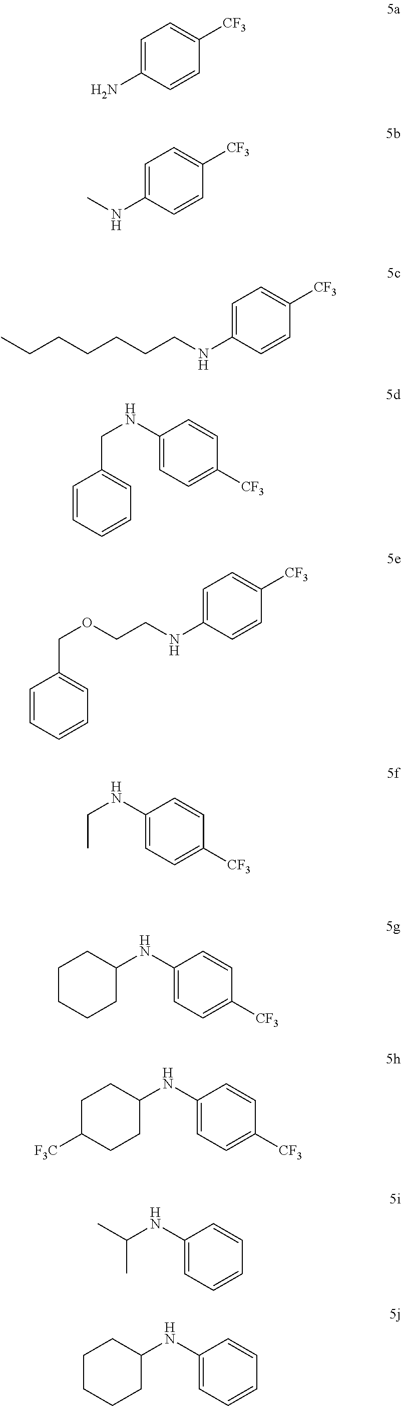

Amines 5a, 5b, 5i, and 5j were purchased from commercial sources.

Amines 5c and 5d were synthesized according to the scheme below.

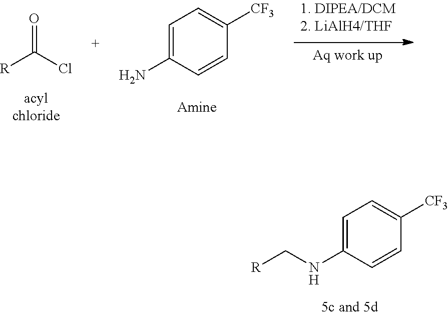

4-(Trifluoromethyl)aniline (1 molar equiv) was dissolved in DCM and to this was added the appropriate acyl chloride (3 molar equivalents) followed by DIPEA (3 molar equivalents). The reaction mixture was stirred at room temperature overnight. Solvent was then evaporated and the crude was dissolved in DCM followed by washing twice with water and brine. The organic layer was then dried with anhydrous sodium sulfate. Solvent was evaporated under reduced pressure to give solid residue which were purified either by trituration or by flash column chromatography using silica gel to give pure amide. Each of the amides obtained were then reduced to amine by reaction one amide equivalent with 10 molar equivalent of LiAlH₄ using an established method. The LiAlH₄ was placed in an ice bath and cold THF was added. This solution was stirred gently and 1 molar equivalent of the amide dissolved in THF was added dropwise. The ice bath was removed and the reaction mixture was stirred overnight at room temperature (12-24 h). Water (3 times LiAlH₄ molar equivalence) was added dropwise followed by 20% (w/v) of NaOH solution (3 times LiAlH₄ equiv) and then water (5 times LiAlH₄ equiv). The solution formed huge gelatinous precipitates and was filtered. The organic volatile part of the filtrate was evaporated, and the aqueous mixture left over was washed with dichloromethane (twice) to remove organic compounds. The organic layer was dried over anhydrous sodium sulfate and solvent was evaporated. The crude was purified to obtain the amines 5c and 5d using flash column chromatography with gradient elution, using hexane and ethyl acetate mixtures. All compounds were completely analyzed and characterized by ¹H NMR and ¹³C NMR.

Compound 5e was synthesized starting from benzyl alcohol as shown in the synthetic scheme 3 below.

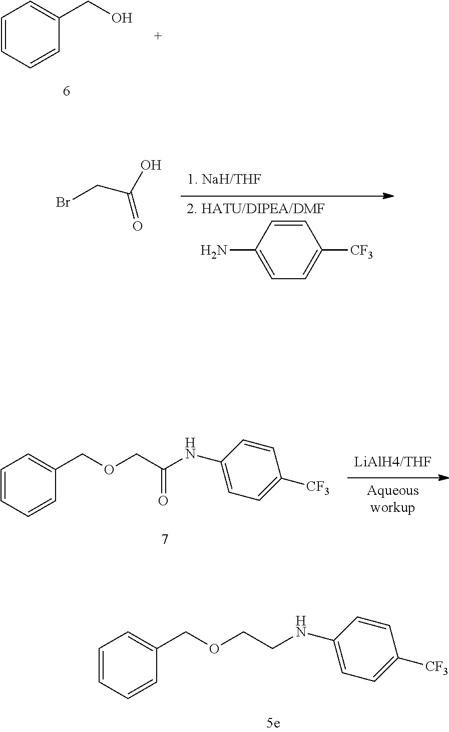

Benzyl alcohol in a round bottomed flask was dissolved in THF and the mixture was placed in ice bath. To this was added NaH portionwise. The mixture was stirred for 5 minutes and then a solution of 2-bromoacetic acid (1 molar equiv) in THF was added dropwise. The ice bath was removed and the mixture stirred overnight at room temperature. All solvent was evaporated under reduced pressure, water was added and concentrated HCl was added to lower the pH to 2-4. The aqueous layer was washed three times with diethyl ether to extract the desired product. The organic phase was dried with anhydrous magnesium sulfate and solvent was evaporated to give a brown oil which was the desired product. The yield was 85%.

This product was dissolved in DCM and DIPEA followed by a solution of HATU in DMF. This mixture was stirred for 5 minutes at room temperature. Then 4-(trifluoromethyl) aniline was added. The mixture was stirred overnight at room temperature. Reaction was stopped and solvent was evaporated under reduced pressure. The crude product was purified using preparative HPLC to give compound 7. Reduction of compound 7 to 5e using LiAlH₄ was done as described above for 5c and 5d. It was completely analyzed and characterized by ¹H NMR and ¹³C NMR.

The amines 5f to 5h were synthesized by reductive amination as shown below.

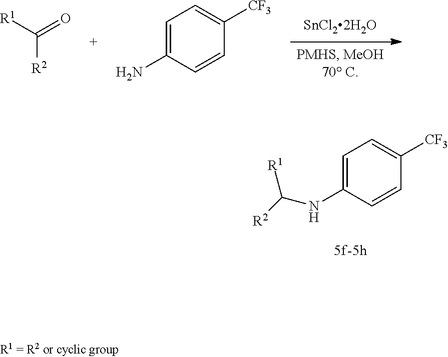

The reaction was done as reported in literature (Nayal et al. J. Org. Chem. 2015, 80(11) 5912-5918) with some modifications. To a stirred solution of SnCl₂·2H₂O (0.2 equivalent) in MeOH was added appropriate ketone compound (1.2 mol equiv), 4-(trifluoromethyl)aniline (1 mol equiv.) and 2.5 mol equiv of PMHS at room temperature. The mixture was placed in oil bath at 70° C. and then refluxed for 4 hours. Heat was removed and the reaction was left to cool down to room temperature. It was filtered under suction and the filtrate was dried over anhydrous sodium sulfate. Solvent was evaporated under reduced pressure to give a crude product which was purified using flash column chromatography with 100% hexanes. All compounds were completely analyzed and characterized by ¹H NMR and ¹³C NMR.

### Synthesis of Compounds

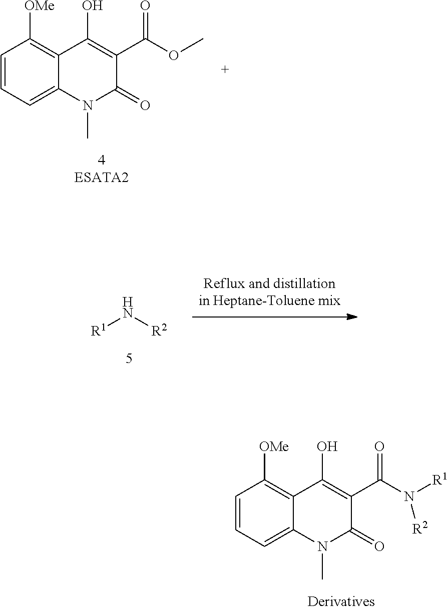

ESATA2 was dissolved in toluene to obtain a clear solution and then a heptane solution of appropriate amine in excess molar equivalence was added. The mixture was heated under reflux. Solvent and volatiles were evaporated by distillation to about 15% solution after 1 hour to remove all the methanol that is formed during this reaction. Then more solvent was added and the same process repeated after another hour. This was done repeatedly every hour over a 5 hour reaction time. The heat was removed after 6 hours and the reaction was stirred at room temperature overnight. Precipitates were formed and this was filtered off. The residue is the unreacted ESATA2. The desired product is in the filtrate. Solvent was evaporated from the filtrate and it was purified by preparative HPLC to obtain each of the derivatives in pure form as described below. Each was fully characterized by ¹H NMR, ¹³C NMR, MALDI-MS, and HPLC. Yield, percent purity, and MALDI-MS data are shown in Table 1 below.

Compounds ESATA25 and ESATA26 were synthesized from ESATA20 as shown below.

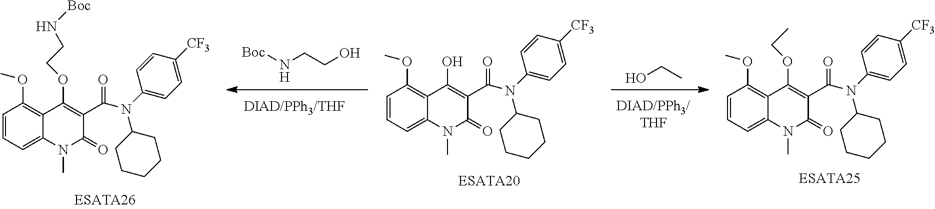

To a solution of ESATA20 in THF was added 2 molar equivalents of DIAD and PPh₃. The reaction was stirred at room temperature for 10 minutes and the respective alcohol was added. The reaction mixture was then stirred at room temperature for 24 hours. Solvent was evaporated and the crude product was purified first with flash column chromatography and then with preparative HPLC to obtained the desired product. Each of the final products (ESATA25 and ESATA26) was fully analyzed and characterized using ¹H NMR, ¹³C NMR, MALDI-MS and HPLC.

Compound ESATA24 was synthesized as shown below.

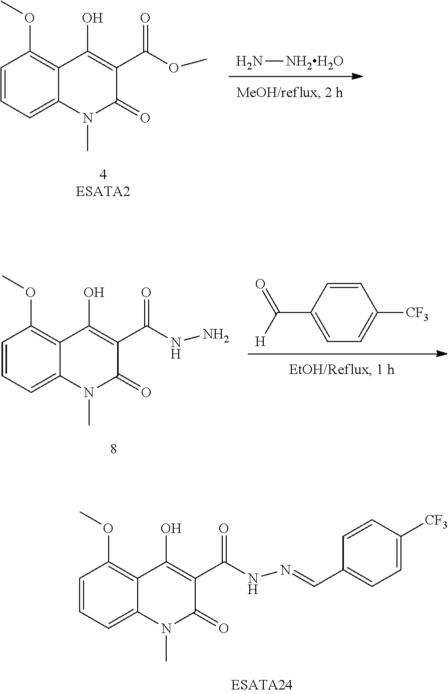

To a solution of 1.0 molar equivalent of ESATA2 in methanol was added excess hydrazine hydrate (About 10.0 molar equivalent). This mixture was refluxed for 2 hours. Heat was removed and the reaction was let to cool to room temperature, diluted with water and left to stand for about 12 hours for precipitation to occur. The solid precipitate was filtered and air dried under fume hood to give Compound 8. This was used for the next chemical reaction without further purification.

To an ethanolic solution of 1.0 molar equivalent of Compound 8 was added 1.5 molar equivalents of 4-(trifluoromethyl)benzaldehyde. This reaction mixture was refluxed for 1.0 hour. Heat was removed and reaction cooled followed by filtration. The residue was purified using preparative HPLC to obtain the desired product.

TABLE 1
Compounds and Characterization Data

| | | Purity | | |
|---|---|---|---|---|
| | | (%) | Yield | MALDI-MS |
| Compound | Structure | (HPLC) | (%) | (M + 1) |
| ESATA11 | 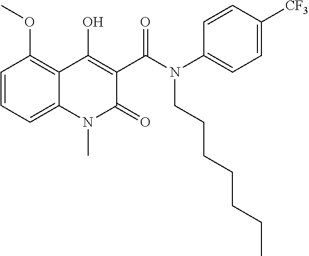 [SMILES: CCCCCCCN(C(=O)c1c(O)c2c(OC)cccc2n(C)c1=O)c1ccc(C(F)(F)F)cc1] | 95 | 63 | 491.75 |
| ESATA14 | 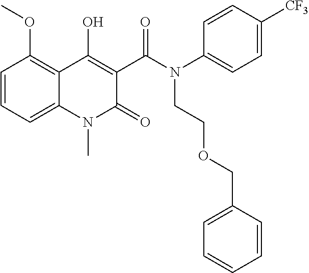 [SMILES: COc1cccc2c1c(O)c(C(=O)N(CCOCc1ccccc1)c1ccc(C(F)(F)F)cc1)c(=O)n2C] | 92 | 66 | 527.69 |
| ESATA18 | 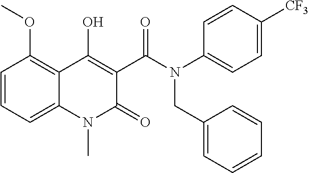 [SMILES: COc1cccc2c1c(O)c(C(=O)N(Cc1ccccc1)c1ccc(C(F)(F)F)cc1)c(=O)n2C] | 96 | 68 | 483.81 |
| ESATA20 | 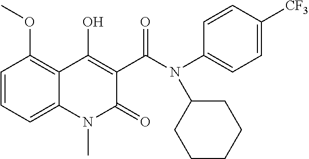 [SMILES: COc1cccc2c1c(O)c(C(=O)N(c1ccc(C(F)(F)F)cc1)C1CCCCC1)c(=O)n2C] | 95 | 63 | 475.70 |
| ESATA22 | 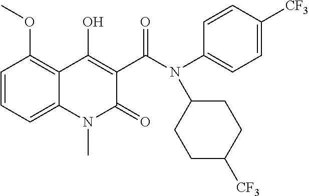 [SMILES: COc1cccc2c1c(O)c(C(=O)N(c1ccc(C(F)(F)F)cc1)C1CCC(C(F)(F)F)CC1)c(=O)n2C] | 97 | 72 | 543.58 |
| ESATA24 | 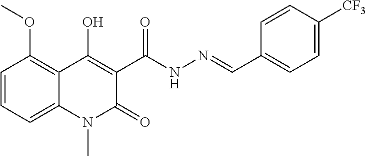 [SMILES: COc1cccc2c1c(O)c(C(=O)N/N=C/c1ccc(C(F)(F)F)cc1)c(=O)n2C] | 90 | 50 | 420.80 |
| ESATA25 | 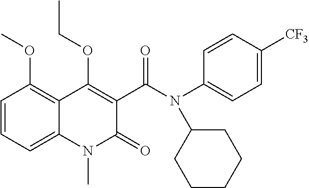 [SMILES: CCOc1c(C(=O)N(c2ccc(C(F)(F)F)cc2)C2CCCCC2)c(=O)n(C)c2cccc(OC)c12] | 95 | 58 | 503.72 |
| ESATA26 | 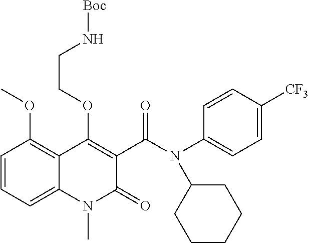 [SMILES: COc1cccc2c1c(OCCNC(=O)OC(C)(C)C)c(C(=O)N(c1ccc(C(F)(F)F)cc1)C1CCCCC1)c(=O)n2C] | 90 | 54 | 618.72 |
| ESATA27 | 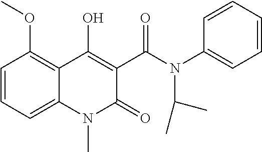 [SMILES: COc1cccc2c1c(O)c(C(=O)N(c1ccccc1)C(C)C)c(=O)n2C] | 98 | 77 | 367.90 |
| ESATA28 | 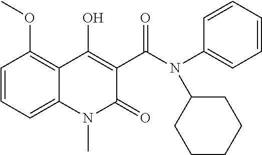 [SMILES: COc1cccc2c1c(O)c(C(=O)N(c1ccccc1)C1CCCCC1)c(=O)n2C] | 96 | 76 | 407.85 |
| ESATA30 | 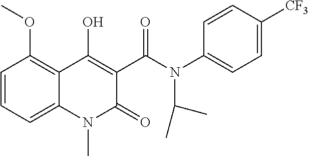 [SMILES: COc1cccc2c1c(O)c(C(=O)N(c1ccc(C(F)(F)F)cc1)C(C)C)c(=O)n2C] | 95 | 79 | 435.68 |

### Example 2

### Assays and Characterization

### Materials and Methods

Prostate Cancer Models. The source, history, and characteristics of the human prostate cancer cell lines and PDXs used, as well as cell culture conditions for their in vitro maintenance and the in vivo protocol for xenograft growth in triple immune-deficient NSG (i.e. NOD.Cg-PrkdcScidIl2rgtmlWji/Szj), adult (>8 week old) male mice obtained from the Sidney Kimmel Comprehensive Cancer Center (SKCCC) Animal Core Facility are described previously (Isaacs 2013; Brennen et al. JCI Insight. 2021, 6(8):e146827; Zhu et al. Oncogene 2020, 39(45): 6935-6949). Similar information concerning the TRAMP-C2 and B6CaP mouse prostate cancers originating in a C57BL/6J male mice are described previously (Foster et al. Cancer Res. 1997, 57: 3325-3330; Simons et al. Oncotarget. 2019, 10(64):6845-6854). In vitro growth curves were determined as described (Isaacs 2013). In vivo growth response to oral daily TasQ (10 mg/kg/d) of TRAMP-C2 mouse prostate cancers was evaluated in C57BL/6J syngeneic wild type vs. NOX-2 vs. iNOS null male mice obtained from Jackson Laboratory (Bar Harbor, ME). Growth response to oral daily TasQ (10 mg/kg/d) of TRAMP-C2 and B6CaP mouse prostate cancers was also evaluated in S100A9 null C57BL′6J male mice obtained from Dmitry Gabrilovich (Wistar Institute). All lines were mycoplasma negative using the MycoSensor PCR Assay kit (Agilent Technologies) and genetically authenticated within the last 6 months using short tandem repeat profiling conducted by the Johns Hopkins Genetic Resource Core Facility. In vitro growth curves were determined as described (Isaacs 2013). Growth response to daily oral TasQ vs. ESATA-20 (both at 10 mg/kg/d) of Myc-CaP-CR mouse prostate cancer was evaluated in syngeneic FVB/NJ castrate male mice from Jackson Laboratory. Animal studies were conducted according to animal protocols approved by Johns Hopkins Animal Care and Use Committee. Tumor volume measurements were as described previously (id.). In vivo experiments were repeated multiple independent times for each model used with 3-5 mice per treatment grouping.

To evaluate AHR agonist induced thymus regression, groups of five 4-week old FVB male mice from Jackson Laboratory were treated with either vehicle, Linomide, TasQ, or its analogs daily by orally gavage for 2 weeks and then the thymus removed and weighted.

Generation of stable shRNA Knock Down (KD) Variants. Stable shRNA HDAC4, and HIF-1α knockdown (KD) were as described previously (id.). Stable shRNA AHR KD as described previously (id.) using ATCCACAGTCAGCCATAATAA (SEQ ID NO: 1) as the targeting sequence.

Western Blot Analysis. Analysis and quantification of protein expression via immunoblot was performed as previously described (Brennen 2021). All assays were performed with total cell lysates from an equal number (i.e., 100,000) of cells loaded per lane for accurate comparisons across cell lines and conditions.

Oral Drug treatment. TasQ at the indicated dose was initially dissolved with equal molar 1 N sodium hydroxide. This solution was diluted with sterile water and the mixture stirred for 1 hr. The pH was then adjusted with 1 N HCl to 8.9 and 200 μL given daily via oral gavage. Benzo[a]pyrene (Sigma-Aldrich) and each of the TasQ analogs were given orally at the indicated daily dose in 200 μL of 1% carboxymethyl cellulose, 0.1% Tween-80, 5% DMSO.

Pharmacokinetic (PK) Analysis. Plasma or serum samples; 50 uL of plasma or serum was transferred into a 1.7 mL microfuge tube. Three volumes of acetonitrile containing 0.1% TFA was added and the sample was vortexed for 1 to 3 minute, then centrifuged at 13,000 RPM in a microcentrifuge. 120 uL of the resulting supernatant was loaded into a sample loop with a cut off volume of 100 uL and injected into the HPLC onto a C18 reverse phase column. The HPLC system was a WATERS 600 series quaternary system with a 2487 dual wavelength detector set at 215 nm and 332 nm. Mobile phase A was 5% Acetonitrile/water, 0.1% TFA Mobile phase B was 95% Acetonitrile/water, 0.1% TFA 1.5 mL/min 20% B to 98% B in 10 minutes, hold for 5 minutes return to initial condition for 4 minutes/Retention time for tasquinimod was 11 min, RT for ESATA20 was 13.3 min with no observable interference from endogenous mouse peaks.

Surface Plasmon Resonance (SPR) Binding Analysis. SPR analysis was performed on the Biacore 3000™ system using CM5 sensor chips, certified buffers (HBS-P—10 mM HEPES, 0.15 M NaCl, pH 7.4, containing 0.005% v/v Surfactant P; HBS-EP-HBS-P with 3 mM EDTA) and reagents for immobilization and surface regeneration from Biacore, GE Healthcare, Uppsala Sweden. Evaluation of sensorgrams was performed using the BIA-evaluation Software version 4.1 and GraphPad Prism 4. Buffer of protein reagents were changed to HBS-P on Zeba spin desalting columns (Thermo Scientific) prior to being used in SPR analysis (Isaacs 2013).

A. Binding of wild-type and mutant forms of HDAC4 to immobilized TasQ. TasQ (ABR-215050; mol wt 406.4) was dissolved as a 40 mM solution in water with 1.5 equivalents of NaOH. TasQ with an amino-linker [i.e. —(CH₂)₂O(CH₂)₂—NH₂](ABR-225180) in amide nitrogen position was used for amine coupling on to a Biacore CM5 sensor chip. A 1 mg/mL solution of ABR-225180 in HBS-P buffer was prepared and immobilized on a CM5 chip by injection at a flow rate of 5 or 10 μL/min until a stable response level was reached. Interaction of various HDAC4 forms with immobilized TasQ was studied by injection over this surface in HBS-P buffer containing 10 or 20 μM ZnCl₂. Bound HDAC4 was removed by a pulse of 3 mM EDTA in HBS-P or 0.5 to 6.25 mM NaOH in water. In a second series of experiments, the ability of TasQ in solution to displace this binding was studied by pre-incubation of HDAC4 with TasQ as competitor.

B. Binding of TasQ to immobilized HDAC4 and NCoR1. Different variants of HDAC4 (full-length wild-type and mutant forms and the truncated protein) and full-length NCoR1 were immobilized on a CM5 chip using random amine coupling. Briefly, proteins were dissolved in 10 mM sodium acetate buffer at pH 5.0 or 5.5 and immobilized to the aimed level using the standard protocol from GE Healthcare. Serially diluted TasQ in HBS-P buffer with 10 or 20 μM Zn was injected (for 2 or 3 min at a flow rate of 30 μl/min) either directly or after a 1^{st} injection of 1 mM DTT to reduce cysteines in the zinc conformation site in HDAC4. Regeneration was made with a short pulse of HBS-EP.

C. Inhibition of HDAC4-NCoR1 interaction by TasQ. The interaction between HDAC4 and NCoR1 was studied either by injecting NCoR over immobilized HDAC4 or with an alternative orientation where HDAC4 was passed over a surface with covalently coupled NCoR1. Whether TasQ can inhibit the formation of the functional HDAC4-NCoR1 complex was tested by pre-incubation with NCoR1 or HDAC4.

Computer docking modeling. This was conducted using Schrödinger's molecular modeling environment Maestro (www.schrodinger.com) as described previously (Isaacs 2013).

RNA Sequencing Analysis of In vivo Growing Xenografts. This was performed as described previously (Brennen 2021; Zhu 2020) with read counts normalized per kilo base-pair gene length and per million reads library size (RPKM).

Three Dimensional (3D) Endothelial Sprouting Assay. The assay was performed using a minimum of 3 replicate wells of a 48 well tissue culture plate per drug dose per experiment, repeated 3 independent times in which human umbilical vein endothelial cells (HUVECs) obtained from Lonza Walkersville Inc (Walkersville, MD) attached to gelatin-coated dextran microcarrier bead are embedded in a fibrin matrix as described previously (Brennen et al. Oncotarget 7, 71298-71308 (2016)).

Targeted kinase screen. Inhibition of enzymatic activity of a panel of kinases in the presence of 10 μM ESATA-20 was analyzed by the Kinase Profiling Service at Thermo Fisher Scientific.

Statistical Analysis. Results are representative and expressed as mean+/−SEM. A p-value of T-test or ANOVA test when appropriate, was considered statistically significant.

AHR Binding Assay. Photo-affinity competitive ligand binding to the human AHR was performed as described previously (Brennen 2021).

AHR Agonist Assay. Human Aryl Hydrocarbon Receptor (AHR) Reporter Assay System [Indigo Biosciences (State College, Pa) Cat #IB06001] was used as per the manufacturer's protocol.

HDAC4 Enzymatic Assay. HDAC4 activity of the various recombinant forms used through these studies was assayed using the small non-protein Lys(Tfa)-AMC as described previously (Bottomley et al. J. Biol. Chem. 2008, 283:26694-26704). The specificity of this assay is documented via its inhibition by a small molecule trifluoroacetylthiophene-trifluoromethylketone derivative (TFT-PIP) described previously (id.).

Real-time quantitative PCR (Q-PCR) analysis. Total RNA was extracted by using RNeasy plus mini kit from Qiagen Cat #74134. One microgram of total RNA was reverse transcribed using iScript cDNA synthesis kit from Bio Rad cat #1708891. One fifth of the first strand cDNA reaction was used for Q-PCR amplification Q-PCR was performed in an iCYCLER real-time PCR machine (BioRad) using SYBR-Green chemistry (BioRad). Test gene Ct values were normalized to Ct values of the house keeper gene GAPDH and fold differences, as compared to untreated controls, were calculated. For semi quantitative PCR one fifth of the first strand cDNA reaction was used for PCR amplification and amplified samples run on a 2% agarose gel stained with ethidium bromide. For mouse CYP1A1 the primer sets were FP: 5′ ATT CCT GTC CTC CGT TAC CTG 3′ (SEQ ID NO: 2) and RP: 5′ GTG GCC CTT CTC AAA TGT CC 3′ (SEQ ID NO: 3). For human CYP1A1 primer sets were FP: 5′ CCC AAC CCT TCC CTG AAT G 3′ (SEQ ID NO: 4) and RP: 5′ TTC TCC TGA CAG TGC TCA ATC 3′ (SEQ ID NO: 5).

### Results and Discussion

### TasQ is an Agonistic AHR Ligand

In vitro growth of human prostate cancer cell lines is only modestly inhibited by TasQ (i.e., IC₅₀>50 μM) under optimal normoxic, nutrient, and pH culture conditions independent of their androgen responsiveness (see, e.g., Isaacs et al. Prostate 2006, 66:1768-78). In contrast to such optimized in vitro conditions, cancers outgrow their blood supply in vivo, producing a compromised TME that is hypoxic and nutrient-limited with an acidic pH. Under these compromised TME conditions, growth of prostate cancer xenografts is profoundly inhibited by oral daily dosing with TasQ (see, e.g., Isaacs 2006). This is due to TasQ inhibiting the downregulation of global histone acetylation via inhibition of HDAC4, which decreases overall transcription needed for cancer cell survival and growth in the compromised TME while also inhibiting the transcription of a select group of survival genes (Isaacs 2013). In addition to its direct effects on cancer cells, TasQ's anti-cancer efficacy also involves dose-dependent anti-angiogenic activity on endothelial cells leading to decreased tumor blood vessel density (see, e.g., Isaacs 2006).

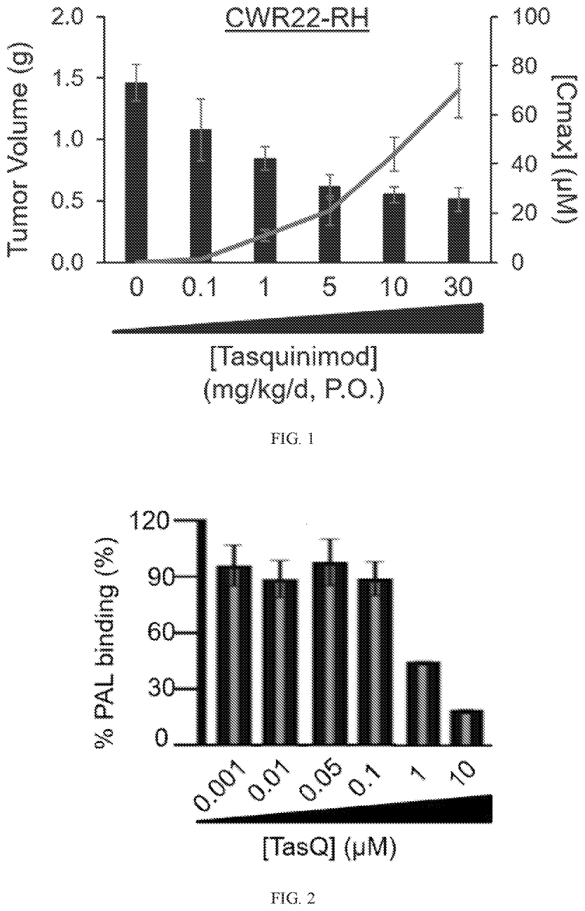

Due to these combined effects, maximal growth inhibition of CWR22-RH, a castration-resistant prostate cancer patient-derived xenograft (PDX), is obtained as maintenance therapy with oral daily dosing of ≥5 mg/kg/d (FIG. 1). PK analysis documents that oral dosing with 5 mg/kg/d of TasQ produces a serum Cₘₐₓ of 20 μM at 1 hr post-dosing (FIG. 1) and a nadir of 1.08±0.21 μM at 24 hrs (Isaacs et al. Oncotarget 2014, 5:8093-8106). Previous studies documented that in vitro treatment of human prostate cancer cells (LNCaP) with 1-10 μM TasQ rapidly (i.e., ≤1 day) induces the transcription of a series of AHR target genes (e.g., Cyp1a1, Cyp1b1, AHRR, TPARP, GDF15) (Olsson 2010). Likewise, upregulation of AHR target genes is also induced in LNCaP xenografts within one day of oral dosing with TasQ at 10 mg/kg (id.).

TasQ is metabolized via microsomal cytochrome P450 (i.e., CYP) enzymes, but inhibition of such metabolism does not affect TasQ's ability to inhibit endothelial cell sprouting in vitro or in vivo growth of human prostate cancer xenografts (Isaacs 2014). This raises the issue of whether the induction of AHR target genes is a result of TasQ or one of its metabolites binds to AHR as a ligand. To test whether TasQ is an AHR ligand, liver cytosol lacking microsomal CYPs was isolated from liver-specific human AHR (hAHR)-expressing transgenic mice. This cytosol was subjected to a rapid (i.e., 20 min) competitive ligand binding assay using a fixed saturating dose of the AHR-specific photo-affinity label (PAL) 2-azido-3-[¹²⁵I]iodo-7,8-dibromodibenzo-p-dioxin with increasing amounts of TasQ. These results document that TasQ is a competitive ligand for human AHR with an EC₅₀ of 1 μM (FIG. 2). As a reference for comparison, the classic polycyclic aromatic hydrocarbon (PAH) AHR agonist, benzo[a]pyrene (B[a]P), has an EC₅₀ value of 1 μM for binding to hAHR in this PAL assay (Flaveny, et al. Mol. Pharmacol. 75, 1412-1420 (2009)).

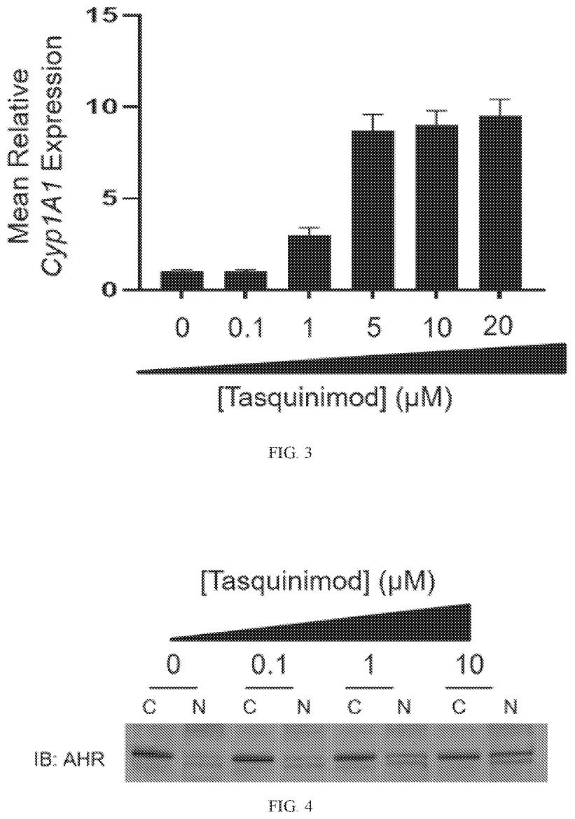

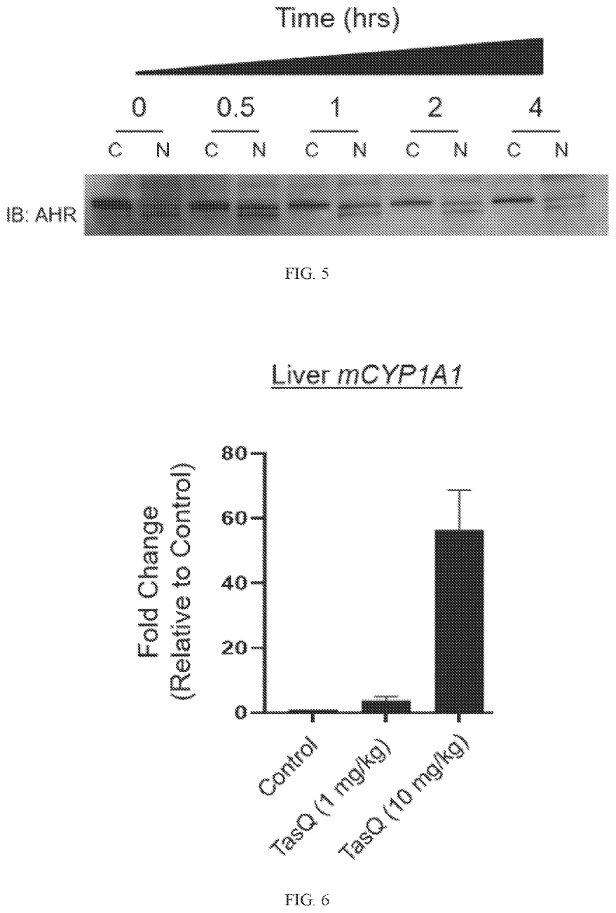

To determine whether such ligand binding is agonistic vs. antagonistic, a human cell-based luciferase reporter gene assay functionally driven by an AHR-responsive promoter was utilized. These studies document that TasQ is a dose-dependent AHR agonist with an EC₅₀ value of 1.0+/−0.2 μM. AHR potency of TasQ in this reporter assay is consistent with the in vitro dose-response induction of Cyp1a1 transcription in LNCaP cells (FIG. 3). Further confirmation that TasQ is an AHR agonist is provided by its dose-response ability to induce rapid (i.e., <30 min) translocation of the AHR from the cytosol to the nucleus of LNCaP cells (FIG. 4). Also characteristic of agonistic binding by TasQ is the subsequent rapid (i.e., ˜2-4 hrs) downregulation of the AHR protein, which is stereotypically induced by agonist binding (Davarinos et al. J. Biol. Chem. 274, 28708-28715 (1999)) (FIG. 5). The systemic in vivo AHR agonist ability of TasQ is confirmed by the fact that oral dosing of 4-week old FVB male mice with 10 mg/kg/d over 14 days maintains the serum drug level at >1 μM, which induces Cyp1a1 transcription in the liver (FIG. 6) and decreases thymus weight (72+/−3 vs. 41.6+/−6 mg in vehicle- and TasQ-treated animals, respectively; p<0.05).

### TasQ's Therapeutic Efficacy Against Prostate Cancer Growth is Independent of AHR

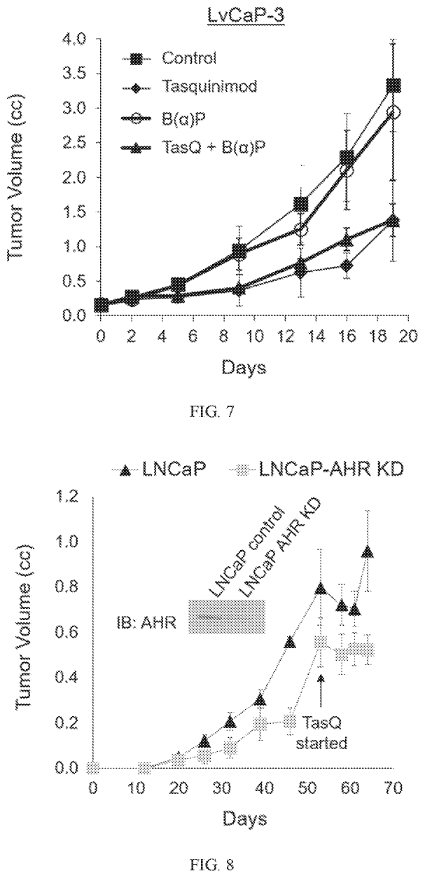

To evaluate whether this AHR agonist ability is required for the MoA responsible for prostate cancer in vivo growth inhibition, a newly established prostate cancer PDX, LvCaP-3, growing in castrate hosts was tested for its response to daily oral dosing with 10 mg/kg TasQ vs. B(a)P alone and in combination (FIG. 7). This PDX is an example of a double-negative prostate cancer (DNPC) xenograft, which lacks both androgen receptor (AR) and neuroendocrine (NE) marker expression, and grows equally well in an intact vs. castrate hosts (Brennen 2021). At this daily dose, B(a)P is a systemic AHR agonist based upon its ability to induce liver Cyp1a1 (Uno et al. Mol. Pharmacol. 65, 1225-1237 (2004)). Like B(a)P, TasQ alone and in combination induces a >50-fold induction of liver Cyp1a1 in treated animals. In contrast, only TasQ alone or in combination with B(a)P inhibits the growth of LvCaP-3 xenografts by ˜⅔ at the end of study, while B(a)P completely lacks therapeutic efficacy (FIG. 7). This is despite oral daily dosing of B(a)P (10 mg/kg/d) for 14 days producing a robust systemic AHR agonist response documented by its ability to decrease thymus weight (>70 vs. 27+/−8 mg in vehicle- and B(a)P-treated animals, respectively; p<0.05).

To further evaluate the role of the AHR in TasQ's MoA, the in vivo growth response to TasQ was compared between castration-sensitive LNCaP vs. LNCaP-AhR shRNA knockdown (LNCaP/AHR-KD) xenografts growing in intact male hosts. This documents that a reduction in AHR protein expression by >90% (FIG. 8 insert), results in a decrease in the growth rate of LNCaP/AHR-KD xenografts compared to the parental LNCaP xenografts in untreated intact male hosts (FIG. 8). Despite this difference, both types of xenografts are equally inhibited when daily oral dosing with TasQ (10 mg/kg/d) is initiated at day 53 post-inoculation (FIG. 8). These combined data document that AHR agonist binding by host cells infiltrating the TME alone (i.e. AHR-KD results) or in combination with prostate cancer cells (i.e. B(a)P results) is not required for TasQ's efficacy.

### TasQ's Mechanism of Action does not Require NADPH Oxidase, iNOS, or S100A9 Expression by Host Cells

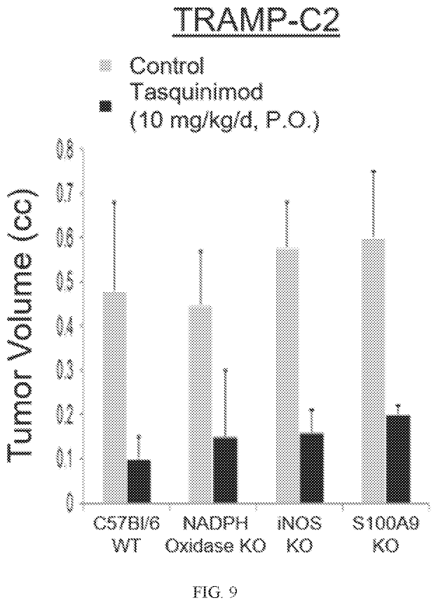

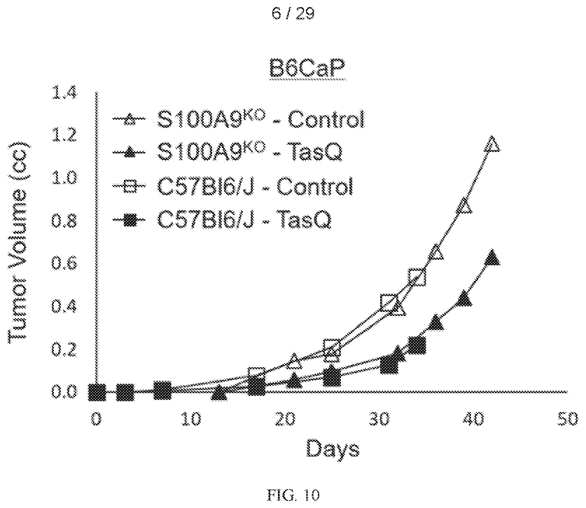

Earlier studies documented that TasQ binds with nanomolar (nM) affinity to S100A9 (Kallberg et al. PLoS One 7, e34207 (2012)). S100A9 is a Ca²⁺-binding pro-inflammatory protein produced by tumor-infiltrating monocytes and macrophages that binds TLR4 and RAGE on tumor-infiltrating myeloid-derived suppressor cells (MDSCs). This binding stimulates reactive oxygen species (ROS) production by NADPH Oxidase-2 (NOX2) and reactive nitrogen species (RNS) produced by inducible Nitric Oxide-2 (iNOS) (Shen et al. Cancer Immunol. Res. 3, 136-148 (2015)). This stimulation is inhibited by the binding of TasQ to S100A9 (Kallberg 2012, Shen 2015). To evaluate the importance of S100A9 inhibition in tumor-infiltrating host cells, the growth response of TRAMP-C2 mouse prostate cancer allografts to daily oral dosing with TasQ (10 mg/kg/d) was compared in syngeneic C57BL/6J wild type hosts vs. NOX2-, iNOS-, or S100A9-knockout (KO) hosts was evaluated (FIG. 9). These results document that expression of neither NOX2, iNOS, nor S100A9 by host cells is required for TasQ's >70% inhibition of tumor growth over a 3-week period. This lack of a requirement for S100A9 in host cells for TasQ's efficacy is reproduced in an additional, newly established mouse prostate cancer model (i.e., B6CaP (Simons et al. Oncotarget 10, 6845-6854 (2019)) also grown in syngeneic C57BL/6J S100A9-KO mice (FIG. 10).

### TasQ Binding Requires Full-Length (fl) HDAC4 Protein in its Open, not Dosed, Conformation

TasQ is an allosteric inhibitor of HDAC4 function (Isaacs 2013). HDAC4 has an N-terminal association domain at amino acid (AA) 68-208, which contains a self-dimerization subdomain at AA 68-155 and a repressive Myocyte Enhancer Factor-2 (MEF-2) binding subdomain at AA 168-184 (Backs et al. Mol. Cell. Biol. 28, 3437-3445 (2008); Martin et al. Oncogene 26, 5450-5467 (2007)). Additionally, there is a nuclear localization signal (NLS) at AA244-279, a nuclear export signal (NES) at AA 1051-1084, and serines at AA 246, 467, and 632 whose phosphorylation is required for binding 14-3-3, restricting localization to the cytoplasm; in addition to a sumoylation site at Lysine 559 (id.). Furthermore, there is an HDAC domain at AA 648-1051, which includes an activating HIF-1α binding domain at AA 822-1051; and a nuclear export signal (NES) at AA 1051-1084 (id.). While HDAC4 lacks intrinsic DNA-binding activity, it selectively binds a subset of client transcription factors as part of either repressive (e.g., MEF2) or stimulatory (e.g., HIF-1) complexes at specific promoter and enhancers (Di Giorgio et al. Mol Cell Biol. 2013, 33(22): 4473-91; Geng et al. J. Biol. Chem. 2011, 286(44): 38095-38102).

Within its C-terminal HDAC domain (AA 648-1051), there is a zinc-bound catalytic domain (ZCD) involving AA 802-950; however HDAC4 is enzymatically inactive against classic acetylated protein substrates (Bottomley 2008; Park et al. Nucleic Acids Res 46, 11776-11788 (2018)). Also within the HDAC domain is a zinc-bound regulatory domain (ZRD) (AA 648-751). The ZRD has two alternative conformations (id.). When Zn²⁺ is coordinated by C667, C669, H675, and C751, the HDAC4 is in an active “closed” conformation which allows binding of the transcriptional co-repressor, NCoR/HDAC3 complex via the RD3 domain of NCoR1 at the rim of the ZCD entry site of HDAC4 (id.). In this HDAC4/N-CoR/HDAC3 complex, HDAC3 is active and deacetylates client proteins tethered to the complex via binding to HDAC4 (Martin 2007). Alternatively, when the Zn²⁺ in the ZRD is coordinated by H665, C667, H678, and C751, the ZRD is shifted to an inactive “open” conformation unable to bind N-CoR/HDAC3 and thus not able to deacetylate HDAC4 bound client proteins (Bottomley 2008, Park 2018).

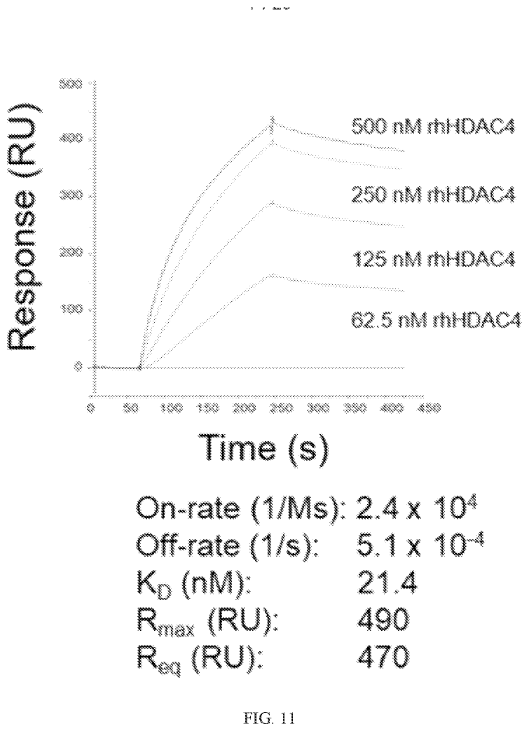

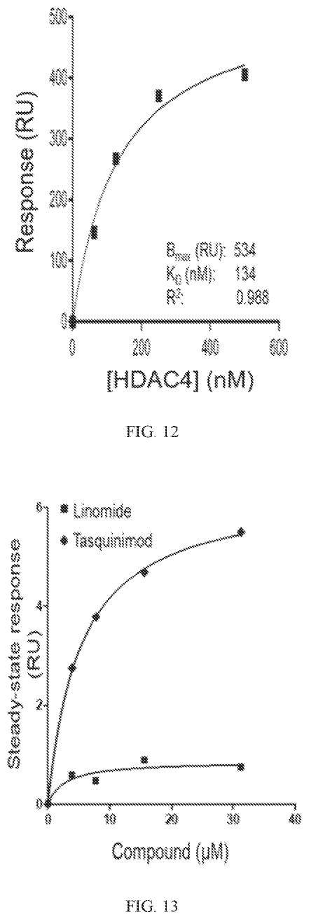

To evaluate the conformational requirements for TasQ's binding to HDAC4, surface plasmon resonance (SPR) analysis was utilized. To maximize conformational flexibility, TasQ is immobilized onto the surface of a Biacore CM5 sensor chip via an amino-linker [i.e. —(CH₂)₂—O—(CH₂)₂—NH₂] in its amide nitrogen position. Using such amine coupling for SPR-binding analysis documented that immobilized TasQ binds in a Zn⁺² dependent saturable manner with a 1:1 stoichiometry with fl-HDAC4 protein in solution with an affinity in the nanomolar range (K_{D}˜20 nM calculated from the on- and off-rates (FIG. 11) or K_{D}˜130 nM from the steady state dose-response curve (FIG. 12)).

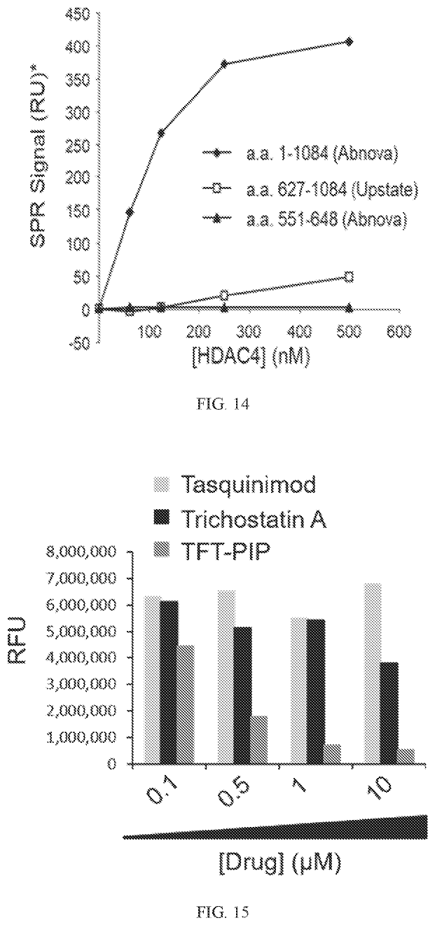

To confirm these results, SPR-binding analysis was repeated, but with the fl-HDAC4 immobilized using random amine coupling onto the surface of a CM5 chip. TasQ in solution demonstrates Zn⁺² dependent saturable 1:1 binding to immobilized fl-HDAC4 with a KD of 4.9+/−1.4 μM (n=4) (FIG. 13). By comparison, Linomide even at >30 μM in solution binds only 10-15% of the immobilized fl-HDAC4 protein despite the presence of Zn⁺² (FIG. 14). TasQ's enhanced affinity for HDAC4 binding compared to Linomide is consistent with its >25 fold increase in anti-tumor efficacy (Isaacs 2010; Isaacs 2006). The fact that the KD for binding of soluble TasQ to immobilized fl-HDAC4 is higher than the KD for binding of soluble fl-HDAC4 with immobilized TasQ is predictable. This is because the conformational dynamics of fl-HDAC4 protein immobilized on the chip are more restrictive due to the multiple random amine coupling than the conformational dynamics of TasQ immobilized via a single amino-linker.

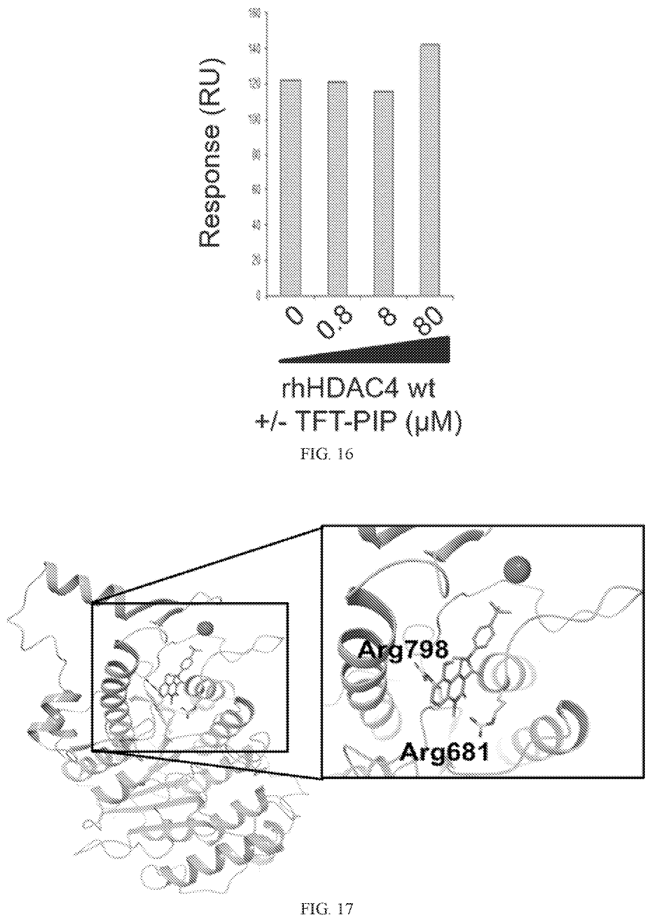

It was previously demonstrated that Zn⁺²-dependent TasQ binding allosterically inhibits formation of HDAC4/NCoR1 (Isaacs 2013). SPR analysis determined that TasQ in solution has no detectable binding to immobilized fl-NCoR1 protein. Thus, TasQ's inhibition of HDAC4/NCoR1 formation involves only its binding to HDAC4. To resolve whether such TasQ binding involves the ZCD vs. ZRD in HDAC4, we took advantage of the fact that while HDAC4 has only marginal deacetylase activity against acetylated lysine-containing proteins, it can deacetylate a small non-protein trifluoroacetamide substrate (a.k.a., Lys(Tfa)-AMC). This reaction is inhibited by a small molecule trifluoroacetylthiophene-trifluoromethylketone derivative [a.k.a., TFT-PIP (FIG. 16)] due to its binding to the catalytic Zn²⁺ in the ZCD, which locks the ZRD in the open conformation (Bottomley 2008). In contrast, TasQ did not inhibit the enzymatic activity of fl-HDAC4 using this Lys(TFA)-AMC substrate (FIG. 16). Importantly, while TFT-PIP locks the ZRD in the open conformation, it does not prevent TasQ binding of fl-HDAC4 (FIG. 17). These results document that TasQ binds the open conformation of fl-HDAC4 at a site other than the ZCD. This is consistent with our previous demonstration that TasQ binds the fl-C669A/H675A double mutant HDAC4 in which the ZRD is also locked in an open conformation (Isaacs 2013).

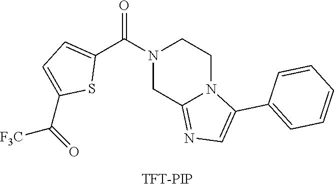 [SMILES: O=C(c1ccc(C(=O)C(F)(F)F)s1)N1CCn2c(-c3ccccc3)cnc2C1 ; "TFT-PIP"]

Unlike the situation for fl-wild type or the fl-C669A/H675A double mutant, the truncated C-terminal HDAC domain (AA 614-1084) containing both the ZCD and ZRD has low and non-saturable binding to immobilized TasQ, and there is no binding to an AA 551-648 HDAC4 fragment (FIG. 14). These results are significant since energy-minimization modeling predicts that TasQ binds to a specific allosteric site only present within the open, not the closed, conformation of the ZRD between AA 612-800 (Bratt et al. Br. J. Cancer 101, 1233-1240 (2009)). Modeling predicts that R681 and R798 within the open conformation of the ZRD region are critically important for TasQ binding (FIG. 17) (Isaacs 2013). This suggests that acquisition of the open conformation needed for TasQ binding to R681 and R798 requires cooperativity with N-terminal sequences (i.e. AA 1-612) present in the full length, but not in the truncated C-terminal HDAC domain (AA 614-1084). Such cooperativity is consistent with the presence of a self-dimerization domain in the N-terminal of fl-HDAC4 located between AA 99-179 and thus missing in the truncated C-terminal HDAC domain (AA 614-1084) (Backs 2008).

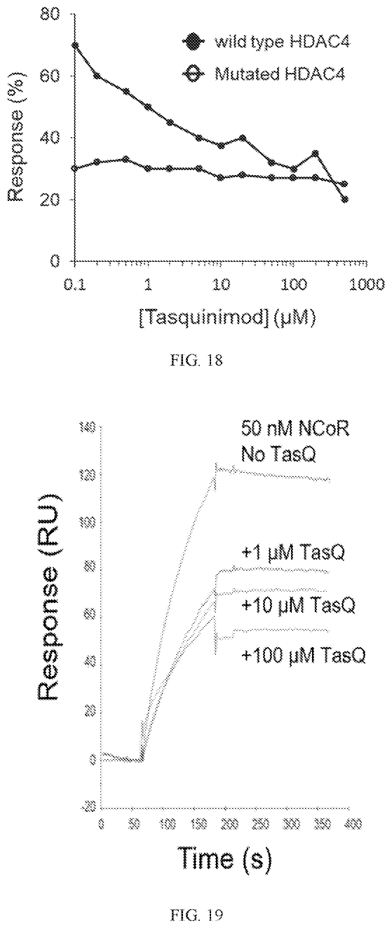

To address this possibility, a competitive binding assay was developed based upon the ability of TasQ in solution to compete with immobilized TasQ on the SPR-chip for binding to fl-HDAC4. These results document that the concentration of TasQ in solution needed to reduce binding of fl-wild-type HDAC4 to the immobilized TasQ by 50% (i.e. IC₅₀) is 1.3 μM (FIG. 18). In contrast, fl-R681A/R798A double mutant HDAC4 protein has a >66% decrease (p<0.05) in binding immobilized TasQ compared to fl-wild type protein, and binding of this fl-R681A/R798A double mutant HDAC4 protein to immobilized TasQ is not competitively displaced by TasQ in solution (FIG. 18).

### TasQ Inhibits Binding Between HDAC4 and NCoR1

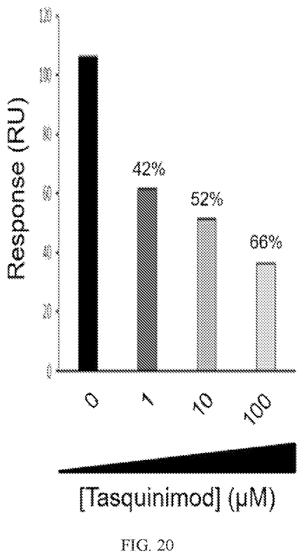

To confirm that TasQ inhibits formation of the functional complex between HDAC4 and NCoR1, fl-NCoR1 in solution was injected over immobilized fl-HDAC4 after pre-incubation with TasQ in solution at concentrations ranging from 1 to 100 μM (FIG. 19). Kinetic analysis of the SPR-sensorgram without competitor determined a high affinity binding (i.e. K_{D}˜0.6 nM) of fl-NCoR1 to immobilized fl-HDAC4, mainly due to a slow off-rate. TasQ in solution inhibits this strong interaction by 50% at 10 μM (FIG. 20).

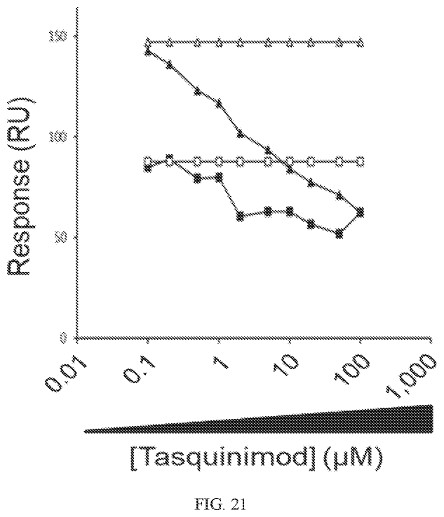

To confirm and extend these results, SPR-analysis was performed with the alternative orientation in which NCoR1 was immobilized on the surface with fl-wild-type vs. fl-R681A/R798A double mutant HDAC4 injected in the presence of TasQ in solution as a competitor. These additional analyses again documented high-affinity binding of the wild-type fl-HDAC4 protein to fl-NCoR1 with such binding being competed by pre-incubation with TasQ in solution (FIG. 21). When the inhibition curve is fit to a sigmoidal dose-response model, the IC₅₀ for TasQ competitive inhibition of fl-HDAC4 binding to immobilized NCoR1 is 1.1 μM which matches the IC₅₀ of 1.3 μM for TasQ binding to immobilized fl-HDAC4 (FIG. 18). In contrast, binding of the fl-R681A/R798A double mutant HDAC4 protein to fl-NCoR1 is reduced by 50% with such reduced binding only slightly inhibited by TasQ in solution (i.e. only <35% inhibition reaching a plateau at 2 μM TasQ) (FIG. 21).

### HDAC4 Disruption Inhibits Tumor Growth Under Hypoxic Conditions

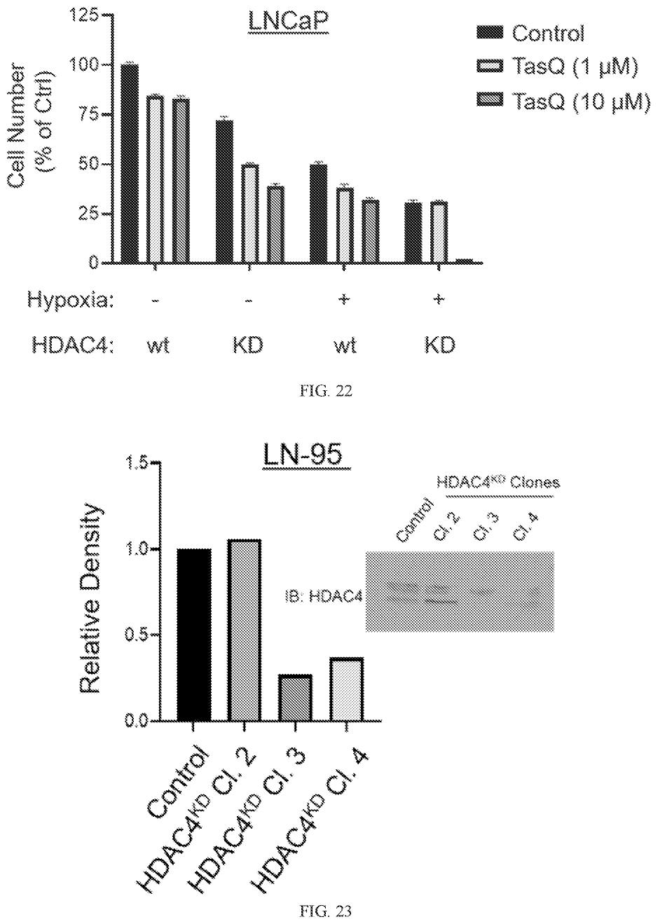

Under optimal normoxic (i.e., 21% 02), nutrient, and pH conditions, in vitro growth of human prostate cancer cell lines is only modestly inhibited by TasQ (i.e., IC₅₀>50 μM) (Isaacs 2013). Similarly, under such optimal conditions, knocking down HDAC4 expression by 50% with shRNA only decreases the growth of LNCaP cells by <25%, which is equivalent to the inhibition by TasQ at 10 μM (FIG. 22). Even under such optimal normoxic conditions, there is a significant enhancement of growth inhibition (i.e., >50%) when HDAC4 KD cells are treated with 10 μM TasQ (FIG. 22). In contrast to normoxic in vitro conditions, cancers outgrow their blood supply in vivo, producing a TME that is hypoxic, nutrient-limited, and acidic (low pH). For example, when prostate cancer xenografts growing subcutaneously reach a size of ˜0.2-0.3 cc, the TME is already hypoxic with a pO₂ of 3.6+/−1.5 mmHg compared to 37.5+/−8.6 mmHg in normal tissue (Dalrymple et al. Prostate 72, 638-648 (2012)). When LNCaP cells are switched from 21% O₂ (i.e., 160 mmHg) to a hypoxic condition of 1% O₂ (i.e., 7.6 mmHg), their growth decreases by 50% (FIG. 22). This hypoxia-induced growth inhibition is enhanced by either knocking down HDAC4 or by treatment with TasQ (FIG. 22). Most significantly, when HDAC4 KD cells are exposed to hypoxia and treated with 10 μM TasQ, the cells die (FIG. 22). These results document that HDAC4 function is critically required for the survival of prostate cancer cells under hypoxic stress.

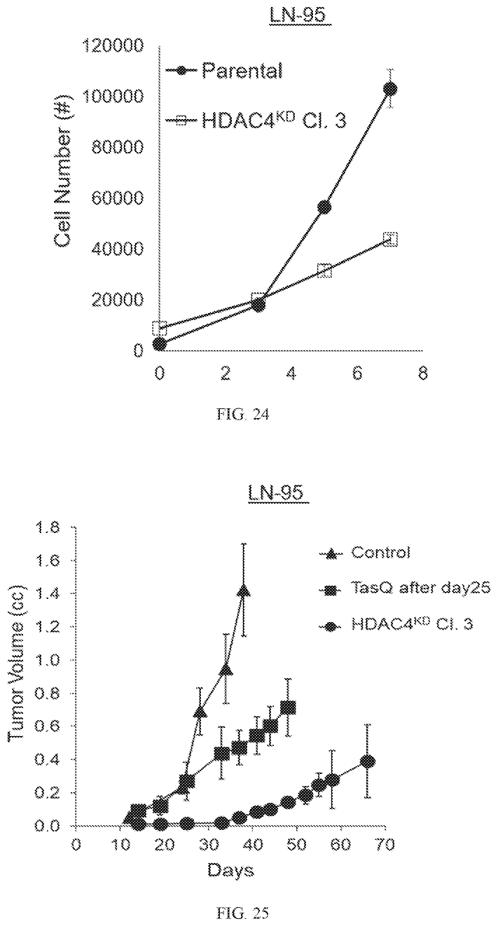

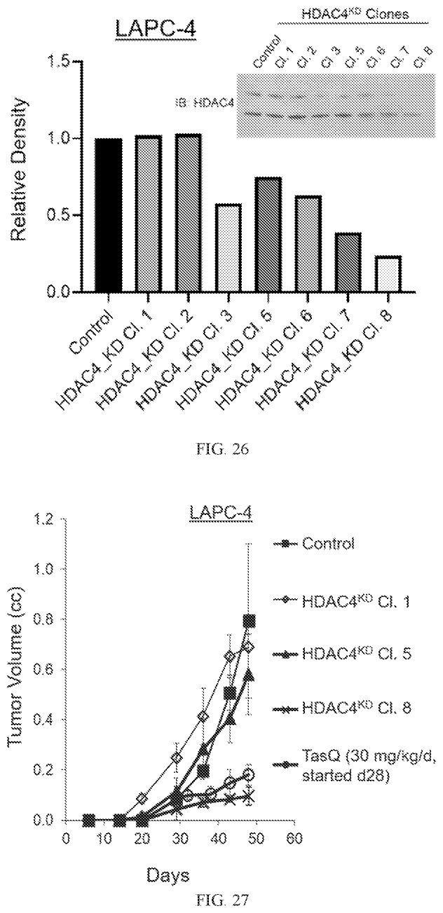

To extend these results, additional prostate cancers lines were evaluated for their growth response to HDAC4 KD alone and in combination with TasQ treatment. In LN-95 cells, a castration-resistant variant of LNCaP, when HDAC4 is KD by >70% [i.e., clone 3 (FIG. 23)], growth is profoundly inhibited under the stressful condition of in vitro growth in androgen-depleted media (FIG. 24) and in vivo growth in castrated hosts (FIG. 25). These in vivo results are consistent with the fact that in prostate cancer xenografts, hypoxia is enhanced >2-fold by daily oral TasQ (10 mg/kg/d) lowering pO₂ in the TME from >3 mmHg to 1.4+/−1.0 mmHg¹⁶. Similarly, when HDAC4 is KD by >75% (i.e., only in clone 8 and not in clones 1 and 5) (FIG. 26), the in vivo growth of castration-sensitive LAPC-4 xenografts in intact male hosts is profoundly inhibited (FIG. 27). Significantly, the growth inhibition of both LN-95 HDAC4 KD xenografts in castrates and LAPC-4 KD xenografts in intact hosts is essentially identical to that of the parental wild type xenografts once daily oral TasQ is initiated (FIGS. 25-27). These combined results document that HDAC4 disruption characteristically enhances hypoxia-induced tumor growth inhibition in vivo in both castration-sensitive and -resistant prostate cancer xenografts.

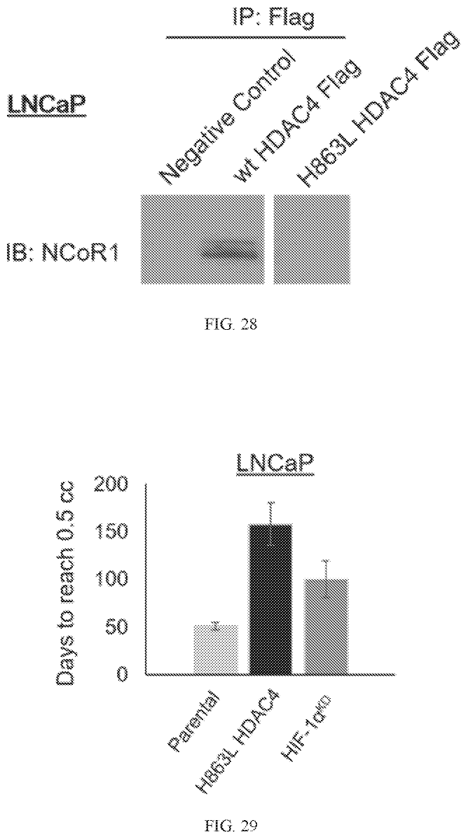

To test if this hypoxia-dependent growth inhibition is due to the loss of HDAC4/NCoR1 formation, LNCaP cells were transfected with a dominant-negative (DN) H863L mutant HDAC4 (Matsuoka, H. et al. Biochem Pharmacol 74, 465-476 (2007)). This mutant protein, like the wild type, lacks protein deacetylase activity, but also like the wild type protein retains enzymatic activity when assayed with the small non-protein Lys(TFA)-AMC substrate. This mutation, however, is DN because it competitively binds to the same subset of transcription factors (e.g., HIF-1α and MEF-2) as wild type HDAC4 (Martin 2007). Unlike wild type, however, the H863L mutant HDAC4 protein does not co-bind NCoR1 (FIG. 28), thus preventing deacetylation by the NCoR1/HDAC3 complex of HDAC4-bound client proteins (Isaacs 2016). This defect results in profound inhibition of in vivo growth, resulting in a 3-fold increase in the time for the xenografts to reach 0.5 cc, which is identical to the growth inhibition by daily oral TasQ (10 mg/kg/d) (FIG. 29).

### TasQ Decreases HIF-1α Protein by Preventing its N-Terminal Lysine Deacetylation

TME hypoxia decreases hydroxylation and acetylation of HIF-1α protein, increasing its cellular level to a point where it activates a transcriptional “pro-angiogenic switch” enhancing tumor angiogenesis and adaptive metabolic survival signaling (Samanta et al. Biochim Biophys Acta Rev Cancer 1870, 15-22 (2018)). This hypoxia-induced upregulation of HIF-1α protein is an early event in human prostatic carcinogenesis associated with poor clinical outcome and involves a decrease in HIF-1α acetylation (see, e.g., Isaacs 2013). Under normoxic conditions, HIF-1α is acetylated on its first five N-terminal lysine residues (lysine 10, 11, 12, 19, and 21), which destabilizes HIF-1α and enhances its proteosomal dependent degradation (Geng 2011). In addition, these N-terminal acetylated lysines are within HIF-1α's DNA binding domain, and while not preventing HIF-1α/HIF-1β (a.k.a., ARNT; aryl hydrocarbon receptor nuclear translocator) heterodimerization (Jiang et al. J Biol Chem 271, 17771-17778 (1996)), they disrupt DNA binding of this heterodimer to hypoxia response elements (Michel et al. Biochim Biophys Acta 1578, 73-83 (2002).

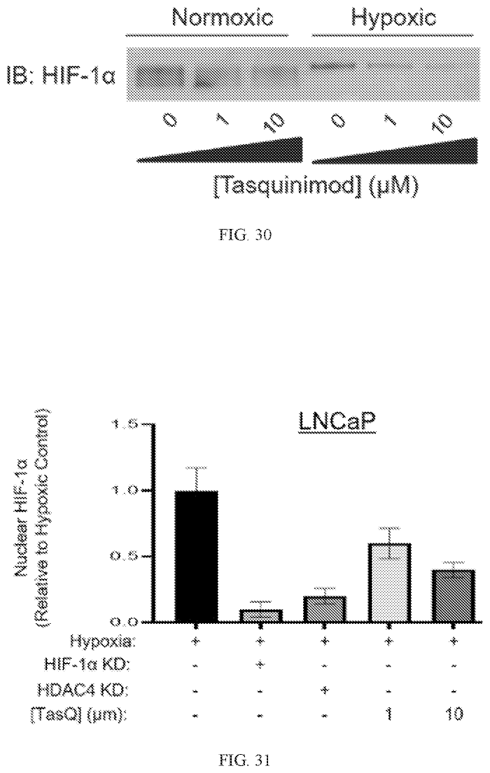

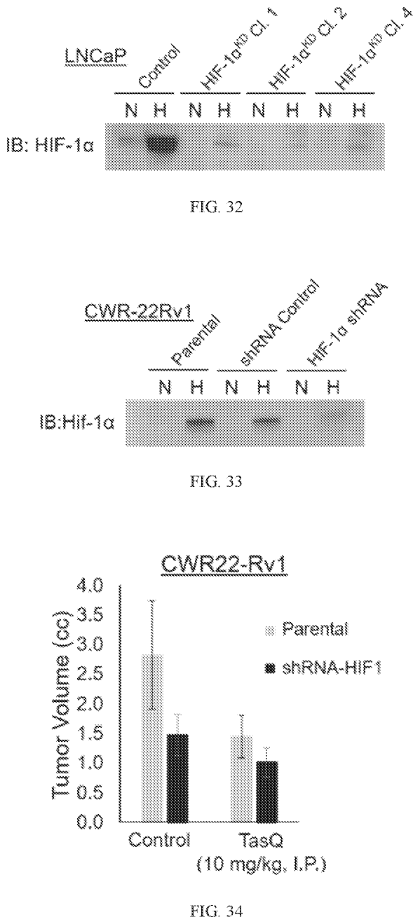

HE-1α is an HDAC4 client (see, e.g., Isaacs 2013) binding between AA 603-788 in its inhibitory domain to HDAC4 (Seo et al. FEBS Lett 583, 55-604 (2009)). As a client, the HDAC4/NCoR1/HDAC3 complex deacetylates the N-terminal lysines, which stabilizes and thus activates HIF-1α as a transcription factor (see, e.g., Isaacs 2013). In LNCaP cells under normoxic conditions, HIF-1α protein is low, cytoplasmic, and mostly unphosphorylated; while under hypoxic conditions, it is increased, nuclear, and phosphorylated (i.e., slower migrating band on western blot) (see, e.g., Isaacs 2013) (FIG. 30). HDAC4 KD in LNCaP prostate cancer cells prevents deacetylation-dependent stabilization, resulting in a reduction of nuclear HIF-1α protein (FIG. 31). As predicted based upon its ability to inhibit formation of the HDAC4/NCoR1/HDAC3 complex, TasQ treatment, like HDAC4 KD, decreases nuclear HIF-1α protein levels in LNCaP cells under both normoxic and hypoxic conditions in vitro with an IC₅₀ of 2.5 μM (FIGS. 30-31). The causal importance of decreased HIF-1α protein for in vivo growth was evaluated by knocking down HIF-1α protein expression in LNCaP cells (FIG. 31), and assessing xenograft growth. Such HIF-1α KD results in significant inhibition of LNCaP growth in vivo, resulting in a 2-fold increase in the time for the xenografts to reach 0.5 cc, which is ˜⅔ of the growth inhibition produced by the DN H863L mutant HDAC4 (FIG. 29).

To confirm the generalizability of this finding, HIF-1α expression was KD in CWR-22Rv1 cells (FIG. 33). When these HIF-1α KD cells are xenografted, their growth over a 3 week period is profoundly inhibited compared to controls (FIG. 34) to a point equal to the growth inhibition of parental wild type xenografts in which their HIF-1α protein level has been decreased by daily oral TasQ (10 mg/kg/d) for 3 weeks (FIG. 34). Significantly, the suppressed growth of these HIF-1α KD xenografts in castrate hosts is only modestly enhanced by the addition of daily treatment with TasQ (10 mg/kg/d) (FIG. 34), consistent with the lowering of HIF-1α being a significant part of the MoA of TasQ.

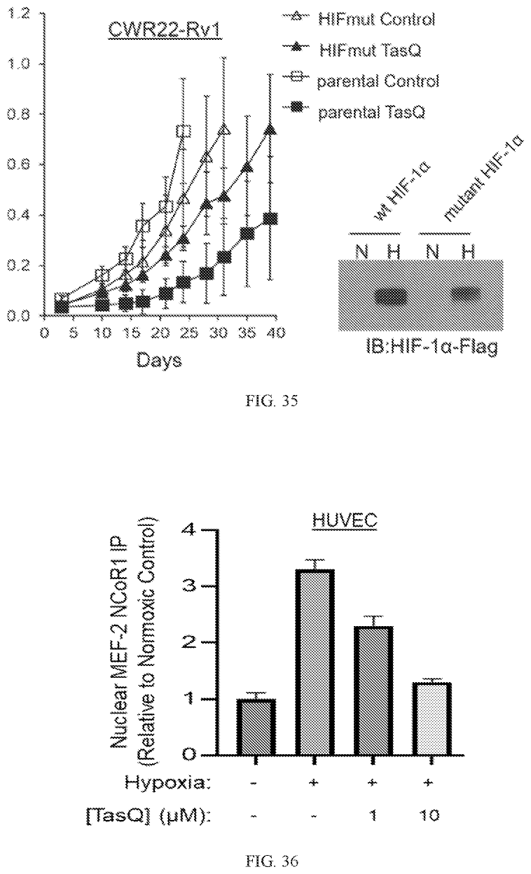

To further query whether decreasing HDAC4-dependent HIF-1α N-terminal deacetylation is a significant component of TasQ's MoA, CWR-22Rv1 cells were transfected with an expression vector encoding flag-tagged HIF-1α in which the N-terminal lysines (K10, 11, 12, 19, and 21) are mutated to arginines (FIG. 35 insert). These mutations prevent acetylation resulting in a gain-of-function (i.e., dominant-positive transcriptional effect) due to stabilization of the protein's half-life⁴¹. When xenografted, these dominant-positive mutant HIF-1α cells grow at a rate similar to the untransfected wild type parental cells (FIG. 35). Unlike the wild type cells whose growth is inhibited by >80% when the host is given daily oral TasQ (10 mg/kg/d), growth of these dominant-positive mutant HIF-1α tumors is only inhibited by <25% in TasQ-treated hosts (FIG. 35).

### TasQ Suppresses HIF-1α Target Genes and Inhibits Repression of MEF-2 Target Genes Needed for Survival in the Compromised Hypoxic TME

RNAseq analysis was performed on CWR22-RH PDX tissue growing in the compromised hypoxic TME of castrated hosts with or without daily oral TasQ (10 mg/kg/d). Such dosing produces optimal CWR22-RH growth inhibition (FIG. 1) consistent with downregulation of the expression of proliferation-associated target genes in addition to the AR and its target genes (Table 1A). In addition, such daily TasQ treatment lowers HIF-1α protein in tumors by >50% (Olsson 2010) suppressing HIF1-dependent cell survival target genes (Table 1B). Significantly, this daily oral dose of TasQ (10 mg/kg/d) maintains blood concentrations of >1 μM (Isaacs 2014). As predicted at this drug level, elevated expression of AHR and a series of its target genes is observed (Table 1C).

While HIF-1α is required for the upregulation of hypoxia survival genes, it is not required for the hypoxia-induced widespread transcriptional repression, which is also needed for enhanced survival in the compromised TME (Denko et al. Nat Rev Cancer 8, 705-713 (2008)). MEF-2 is also a HDAC4 client (McKinsey et al. Trends Biochem Sci 27, 40-47 (2002)). MEF-2 dimers bind to target gene promoters, forming a hydrophobic groove composed of AA 1-78 of each monomer to which HDAC4 binds via its amphipathic helix at AA 168-184 (Han et al. J Mol Biol 345, 91-102 (2005)). When MEF-2 binds, HDAC4 forms a NCoR1/HDAC3 complex, which deacetylates MEF2 and histones within its proximity; thereby, repressing expression of its target genes (McKinsey 2002). When CWR-22Rv1 cells are switched from a 21% to a 1% 02 environment in vitro, there is a 3-fold increase in NCoR1 immunoprecipitated with MEF-2 from nuclear extracts (FIG. 36). Co-treatment with TasQ inhibits this hypoxia-induced increase in nuclear MEF2/HDAC4/NCoR1 formation with an IC₅₀ of 5 μM (FIG. 36). These in vitro results are consistent with gene expression analysis of CWR22-RH PDX tissue from castrate hosts in vivo, which document that daily oral TasQ (10 mg/kg/d) inhibits the binding of NCoR1, and thus HDAC3 to the MEF-2/HDAC4 complex, relieving repression of MEF-2 target genes needed for survival under hypoxic conditions (Table 1B).

TABLE 1
TasQ phenocopies HDAC4 knockdown suppression
of HIF-1α target gene transcription
A. Downregulation of proliferation and androgen receptor target genes
(expressed as FPKM +/− SD) in CWR22-RH PDXs in castrate hosts
treated orally with TasQ (10 mg/kg/d) compared to vehicle control

| Proliferation Target Genes | | AR Target Genes | |
|---|---|---|---|
| (Fold Decrease by TasQ) | | (Fold Decrease by TasQ) | |
| MKI67 | −1.7 | AR | −2.0 |
| E2F1 | −2.0 | TMPRSS2 | −1.9 |
| CDC6 | −1.5 | NKX3.1 | −2.3 |
| CDT1 | −2.2 | KLKP1 | −2.3 |
| MCM2 | −1.9 | SLC45A3 | −3.7 |
| MCM3 | −1.6 | | |
| MCM5 | −2.0 | | |
| DTYMK | −1.6 | | |

B. Downregulation of HIF-1 target genes coupled with
upregulation of MEF-2 target gene expression (expressed as
FPKM +/− SD) in CWR22-RH PDXs in castrate hosts treated
orally with TasQ (10 mg/kg/d) compared to vehicle control

| | |
|---|---|
| HIF-1α Target Genes | MEF-2 Target Genes |
| (Fold Decrease by TasQ) | (Fold Increase by TasQ) |

| | | | |
|---|---|---|---|
| IGFBP5 | −18.0 | EDN1 | 15.0 |
| IGF1 | −11.0 | THB5 | 8.0 |
| PTN | −6.1 | S100P | 5.7 |
| LOX | −4.0 | BHLHE41 | 2.9 |
| TTR | −3.8 | CA12 | 2.7 |
| TNC | −2.5 | CEACAM1 | 1.8 |
| BNIP3 | −1.9 | NR4A2 | 1.8 |
| IGFBP2 | −1.8 | CAPN2 | 1.6 |
| P4HA1 | −1.8 | NR4A1 | 1.4 |
| HILPDA | −1.7 | | |
| PDK1 | −1.7 | | |
| LDHA | −1.5 | | |
| VEGFB | −1.5 | | |
| ALDOC | −1.4 | | |

C. Upregulation of AHR target genes (expressed as FPKM +/−
SD) in CWR22-RH PDXs in castrate hosts treated orally
with TasQ (10 mg/kg/d) compared to vehicle control
AHR Target Genes
(Fold Increases by TasQ)

| | |
|---|---|
| CYP1B1 | 10.0 |
| AGR2 | 5.5 |
| TIPARP | 3.4 |
| AHR | 3.0 |
| NQO1 | 2.9 |
| GDF15 | 2.3 |

### TasQ's Anti-Angiogenic MoA Involves Inhibiting Repression of MEF-2 Target Genes

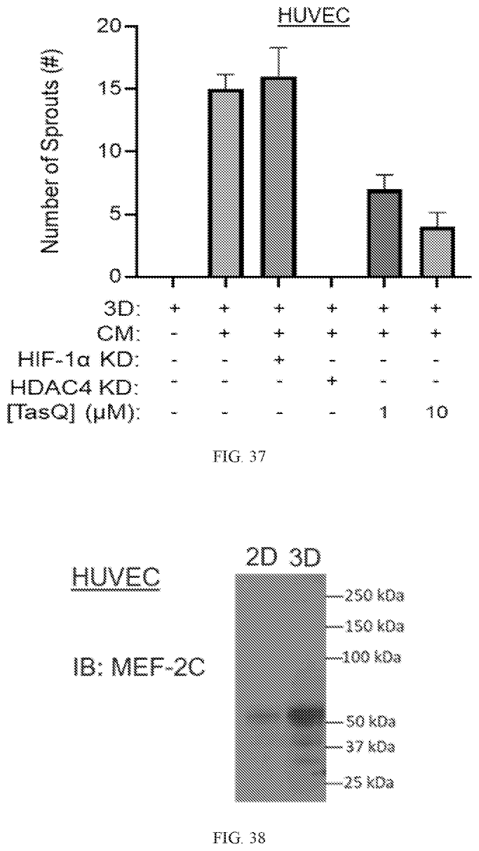

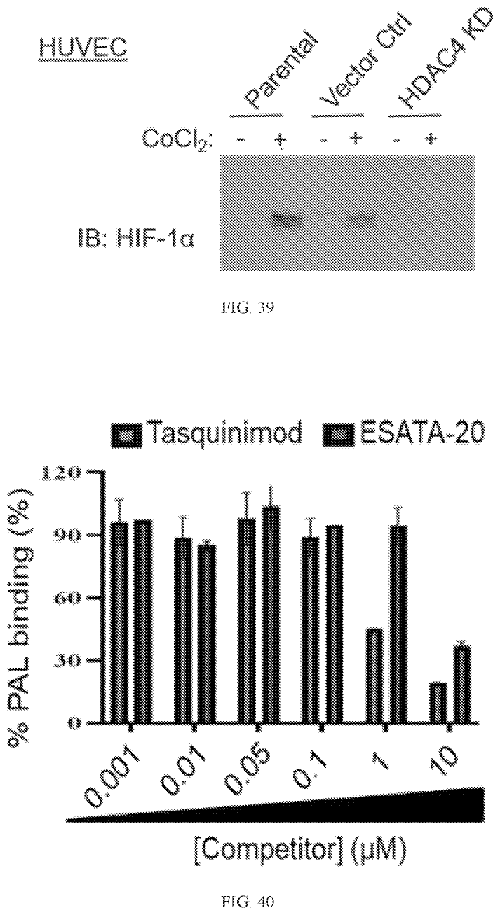

Besides its therapeutic effects on cancer cells, TasQ's anti-cancer efficacy also involves its dose-dependent anti-angiogenic activity (see, e.g., Isaacs 2006, Olsson 2010, Dalrymple 2012), Isaacs 2013, Isaacs 2014, Brennen 2016). To clarify the MoA for this anti-angiogenic activity, a 3-dimensional (3D) endothelial cell sprouting assay was used. In this assay, human umbilical vein endothelial cells (HUVECs) in basal media supplemented with VEGF, EGF, FGF, IGF1 and 2% FBS in standard 2D cultures are attached to collagen-coated beads embedded in a 3D-fibrin gel in the same growth factor-supplemented media. Under these 3D-basal conditions, the embedded endothelial cells undergo a major transcriptional reprogramming, but despite a 4-5 fold increase in ETSV Proto-Oncogenes 1 and 2 Transcription Factors (Ets-1 and -2) coupled with a 2-3 fold increase in Ets target genes like Flt1 (VEGFR1) and KDR (VLGFR2) (Table 2) they do not sprout (FIG. 37). This lack of sprouting is associated with an upregulation in Mef-2c mRNA (Table 2) and protein (FIG. 38), and its target genes (Table 2) like Alpha 2 Macroglobulin (A2M). These results are consistent with earlier studies documenting that sprouting is inhibited in this 3D-basal media due to enhanced expression of Mef-2c coordinately upregulating expression of A2M (Sturtzel et al. PLoS One 9, e101521 (2014)). When the 3D-fibrin gels are further supplemented with conditioned media (CM) from human dermal fibroblasts (HDF) to provide additional angiogenic factors typically present within the TME A2M (Nakatsu et al. Microvasc Res 66, 102-112 (2003)), expression of the MEF-2c target genes, including A2M, is blocked (Table 2); enabling endothelial cell proliferation and invasion into the gels (i.e., “sprouting”); eventually forming canalized and anastomosing neovascular tubes within 7 days (FIG. 37). Importantly, 3D-sprouting occurs (FIG. 37), even when HIF-1α expression is KD in the HUVEC cells (FIG. 39). In contrast, as little as a 60% KD in HDAC4 completely inhibits HUVEC sprouting in this 3D-assay despite the addition of HDF-CM (FIG. 37). Similarly, when these CM-supplemented 3D cultures are treated with TasQ (10 μM), the downregulation of Mef-2c target genes like A2M in the embedded endothelial cells is inhibited (Table 2) and sprouting prevented with an IC₉₀ of 10 μM (FIG. 37).

TABLE 2
mRNA expression normalized as FKPM of HUVEC in 2D vs. 3D-fibrin
gels with or without addition of human dermal fibroblast (HDF)-
conditioned media (CM) with or without TasQ (10 μM).

| HUVEC | Gene Expression (FPKM) | | | |
|---|---|---|---|---|
| Culture Conditions: | 2D | 3D-fibrin | | |
| Fibroblast Conditioned Media: | No | No | Yes | Yes |
| TasQ (10 μM): | No | No | No | Yes |
| Ets1 | 14,676 | 58,225 | 26,551 | 72,987 |
| Ets2 | 9,735 | 43,699 | 18,931 | 45,227 |

Ets Target Gene Expression

| | | | | |
|---|---|---|---|---|
| LI1 (VEGFR1) | 13,554 | 62,942 | 42,357 | 106,830 |
| KDR (VEGFR2) | 36,567 | 85,677 | 79,832 | 99,348 |
| Mef-2c Target Gene | 5,225 | 13,840 | 6,370 | 21,217 |
| Expression | | | | |

Mef-2c Target Gene Expression

| | | | | |
|---|---|---|---|---|
| A2M | 4,041 | 220,767 | 5,163 | 257,405 |
| Apinr | 72 | 85,739 | 1,217 | 81,082 |
| Adamt59 | 2,750 | 49,345 | 5,002 | 89,213 |
| SerpinB1 | 1,868 | 3,915 | 1,378 | 4,926 |
| Spry | 1,203 | 8,209 | 2,788 | 11,728 |
| Stc1 | 80 | 3,461 | 64 | 18,619 |
| NR4A1 | 165 | 1,112 | 872 | 1,598 |
| NR4A2 | 12 | 174 | 58 | 297 |

### Third Generation Quinoline-3-Carboxamide Analogs with Reduced AHR Agonism

Combined xenograft and PK results document that the plasma concentration of TasQ required to inhibit HDAC4 binding to NCoR1 sufficiently to suppress HIF-IG transcriptional hypoxic survival signaling and thus maximally inhibit in vivo prostate cancer xenograft growth is >2 μM. Unfortunately, this is higher than the EC₅₀ value of 1 μM for TasQ as an AHR ligand (FIG. 3) as an agonist in the in vitro cell-based ARE-driven luciferase reporter gene assay (Table 3), and as a CYP1A inducer in LNCaP cells (FIG. 6). This means that in the clinical setting, dose-escalation above 1 mg/d of TasQ as needed to reach this >2 μM blood level for optimal anti-cancer efficacy will also enhance unwanted off-target AHR induction. Therefore, a medicinal chemical approach was undertaken to identify third generation quinoline-3-carboxamide analogs with increased HDAC4 inhibition, but with reduced AHR agonism. The approach taken is based upon the earlier SPR analysis documenting that TasQ linked to the surface chip via its aniline-N is a potent binder of HDAC4. Thus, a series of quinoline-3-carboxamide analogs were synthesized focusing upon adduct changes at the aniline-N. These analogs were synthesized as described in Example 1, and were initially screened for their AHR agonist activity using the in vitro human cell-based ARE-driven luciferase reporter gene assay (Table 3). Based upon this primary screen, a subset of analogs was evaluated for their percent inhibition of the growth of CWR22-RH xenografts in castrate NSG male mice vs. the percent thymus regression in intact FVB male mice following daily oral dosing at 10 mg/kg/d over a 3-week period (Table 3).

The compound ESATA3 in Table 3 is the same as amine Compound 5c in Example 1.

TABLE 3
AHR agonist activity of analogs expressed as EC₅₀ values in an in vitro cell-based ARE-
driven luciferase reporter gene assay (n = 4 independent assays/analog) vs. induction
of thymus regression expressed as percent (%) decrease in organ weight in mice (n =
5/group) treated orally for 2 weeks with the respective compound (10 mg/kg/d) vs. inhibition
of in vivo growth of CWR22-RH xenografts in castrate mice treated orally over a 3 week
period with the respective compound [10 mg/kg/d, (n = 5/group)]. ND = not determined.

| | AHR Agonist Response | Thymus Regression (% | CWR22-RH Tumor Growth |
|---|---|---|---|
| Analog | [EC₅₀] (nM +/−SD) | decrease weight +/− SD) | (% inhibition +/− SD) |
| ESATA-4 | 0.5 +/− 0.2 | 46 +/−7 | 86 +/− 9 |
| ESATA-11 | 50 +/− 25 | 45 +/− 4 | ND |
| ESATA-5 | 300 +/− 111 | ND | ND |
| ESATA-24 | 800 +/− 169 | ND | ND |
| ESATA-27 | 850 +/− 198 | ND | <10 |
| TasQ | 1,000 +/− 100 | 47 +/− 8 | 87 +/− 11 |
| ESATA-14 | 1,100 +/− 200 | ND | <10 |
| ESATA-28 | 1,300 +/− 200 | ND | <10 |
| ESATA-30 | 1,400 +/− 100 | 35 +/− 11 | <10 |
| ESATA-20 | 10,200 +/− 1,000 | 8 +/− 3 | 80 +/− 15 |
| ESATA-18 | 10,700 +/− 2,200 | ND | 50 +/− 18 |
| ESATA-22 | 12,000 +/− 5,300 | 8 +/− 4 | <10 |
| ESATA-25 | 10,500 +/− 1,900 | ND | <10 |
| ESATA-26 | >50,000 | ND | <10 |
| ESATA-2 | >100,000 | ND | ND |
| ESATA-3 | >100,000 | ND | ND |
| Benzo[α]pyrene | 200 +/− 39 | 51 +/− 7 | <10 |
| Abiraterone | >50,000 | ND | <10 |

Structure-activity relationship (SAR) analysis shows that, for these particular compounds, to retain potency as a growth inhibitor (GI) of CWR22-RH xenografts but a decreased potency as an AHR agonist: 1) the quinolone-3-carboxamide linked aniline moiety should be present (e.g. ESATA2 and ESATA3—no GI activity); 2) the aniline N adduct should not be too long (e.g. ESATA14, ESATA22—no GI activity) or too short (e.g. ESATA4—high GI activity, but also much more potent AHR agonist; and ESATA27 and ESATA30—no GI activity); 3) the 4-position adduct should not be too long (e.g. ESATA25 and ESATA26—no GI activity); and 4) a para-trifluoromethyl group should be present in the aniline moiety (e.g. ESATA28—no GI activity). Other combinations of substituent groups may also be suitable.

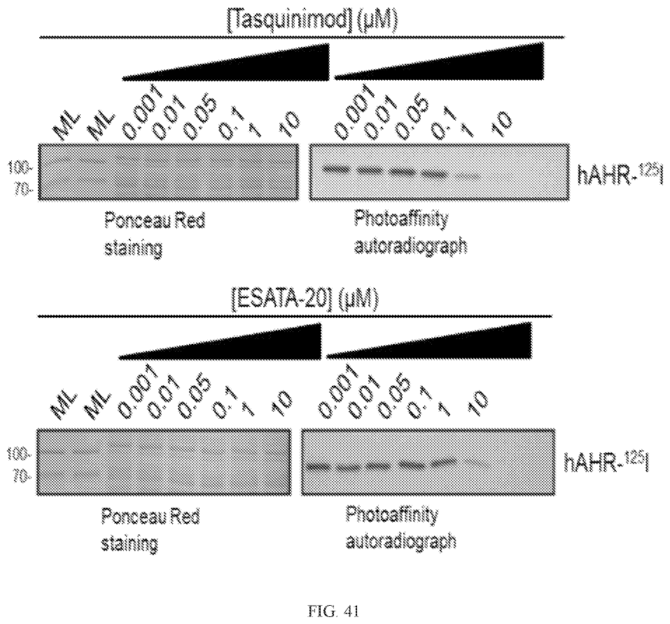

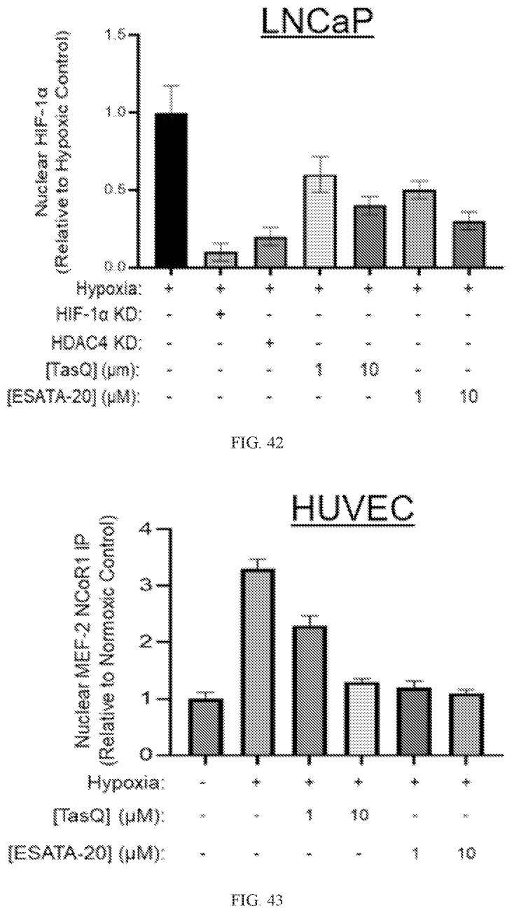

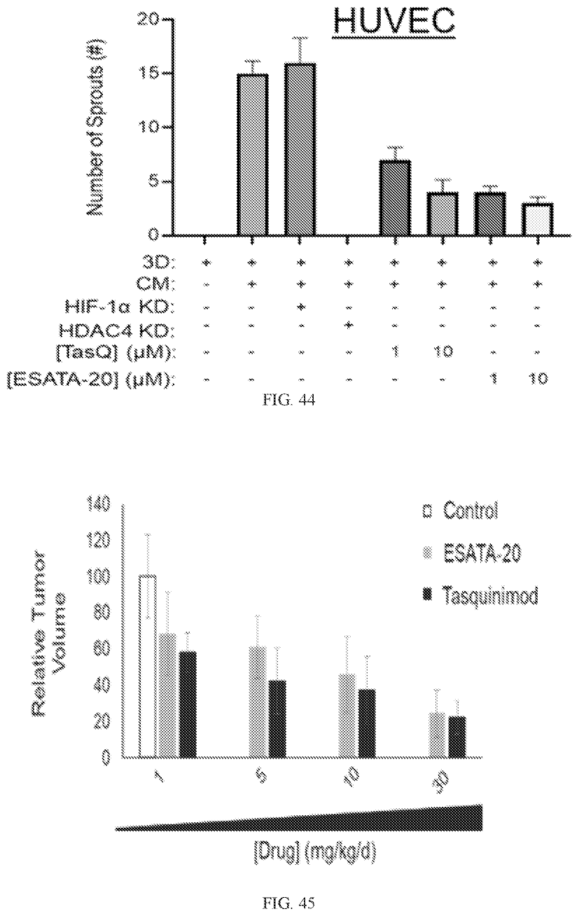

Based upon the SAR, the ESATA20 analog has been identified as a lead 3rd-generation quinolone-3-carboxamide. ESATA-20 has a 10-fold lower binding affinity than TasQ in both the competitive PAL AHR binding assay (FIGS. 40 and 41), and the agonist response in the cell-based AHR-promoter assay (Table 3) (i.e., EC₅₀ of 10 vs. 1 μM for TasQ). In contrast, ESATA-20 is more potent than TasQ at decreasing nuclear HIF-1α protein (i.e., IC₅₀ of 1 vs. 2.5 μM for TasQ) (FIG. 42), nuclear MEF-2 and NCoR1 IP (i.e. IC₅₀<1 vs. 5 μM for TasQ) (FIG. 43), and inhibiting endothelial cell sprouting (i.e., IC₉₀ of 1 vs. 10 μM for TasQ) (FIG. 44).

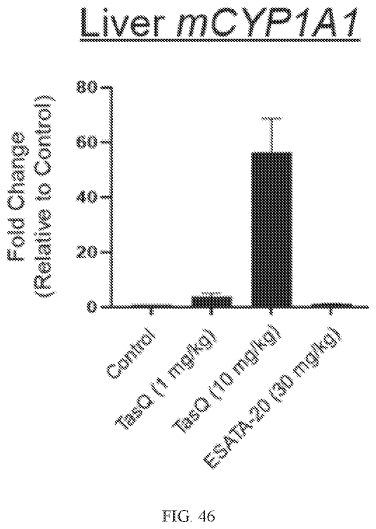

ESATA-20 is less water soluble than TasQ requiring the use of DMSO in a 1:9 ratio with 20% 2-hydoxypropyl-β-cyclodextrin (HPCD) in saline as its vehicle for oral dosing. In this vehicle, ESATA-20 is less bioavailable than TasQ as documented by the fact that a 10 mg/kg oral dose of ESATA-20 produces a serum Cm., of 7.91+/−18 μM vs. a Cₘₐₓ of 44+/−6 μM following an equivalent oral dose (10 mg/kg) of TasQ. Despite this 5.5-fold lower bioavailability, PK analysis documented ESATA-20 has an alpha half-life for tissue distribution of 3 hrs, while the beta half-life for serum elimination is 10-12 hrs with >98% of the drug bound to serum proteins, which is very similar to these serum parameters for TasQ¹⁶. ESATA-20 (10 mg/kg/d) produces the same maximal anti-cancer efficacy as TasQ at the same 10 mg/kg/d dose (FIG. 45). Thus, on a molar basis, ESATA-20 is at least 5-fold more potent than TasQ in inhibiting CWR22-RH xenograft growth. It is also more selective than TasQ as documented by its lack of induction of both liver Cyp1a1 (FIG. 46), and thymus regression (Table 3), even when ESATA-20 is given orally at a 3-fold higher dose (i.e., 30 mg/kg/d).

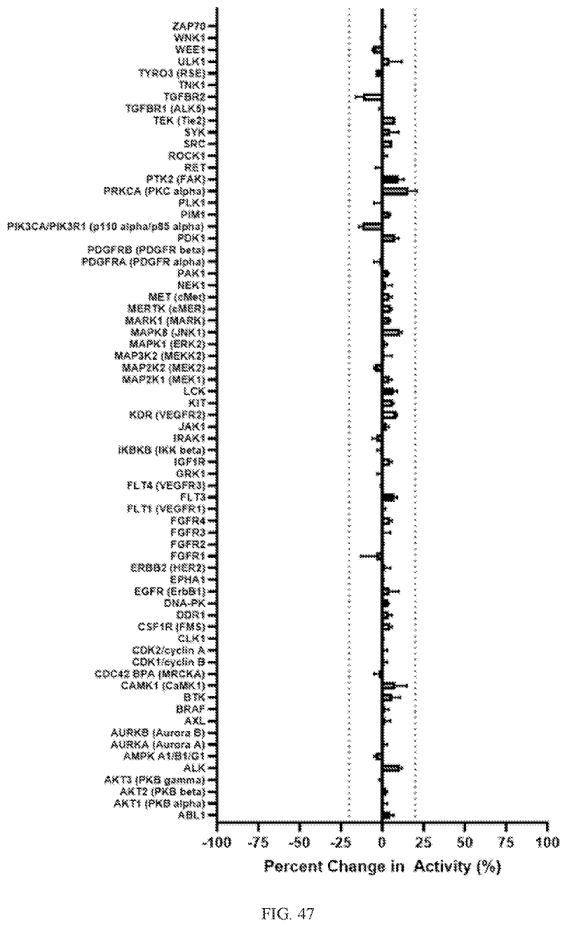

ESATA-20 at a 10 μM dose produced <15% change in activity in any of the 68 kinases tested in a targeted kinase screen representing a wide range of kinase families (FIG. 47). Thus, daily oral dosing of ESATA-20 should be titrated to produce a serum drug level of 2-5 μM; thereby, optimizing its therapeutic efficacy as an HDAC4/HIF1α/MEF-2 disruptor when given to mCRPC patients without inducing the unwanted off-target AHR agonist side effects.

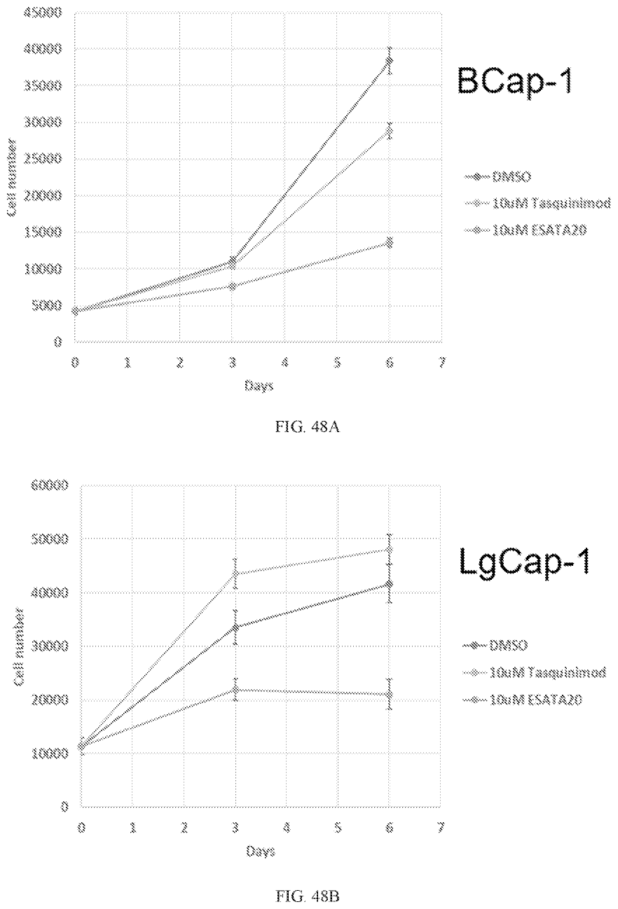

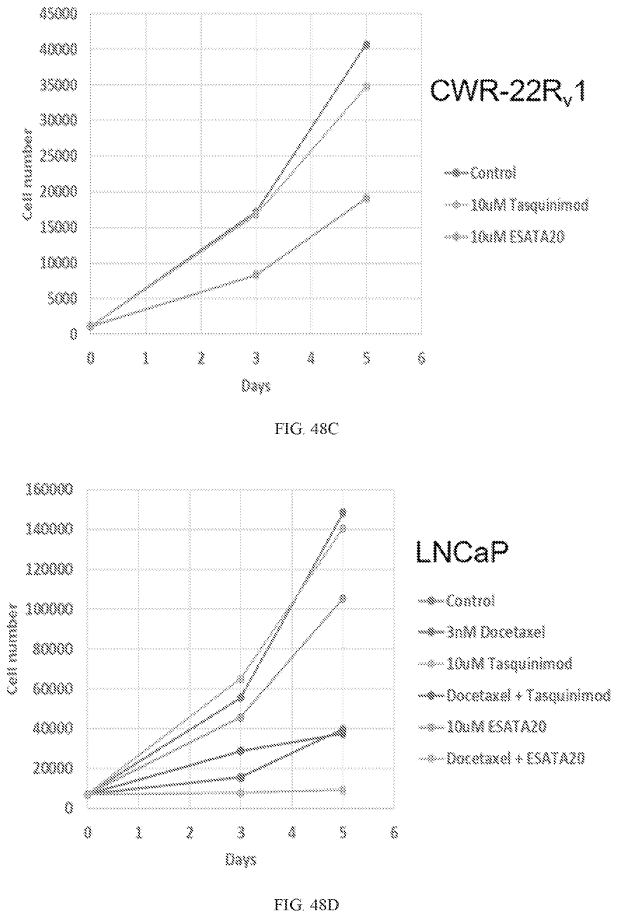

To further compare the therapeutic potency of ESATA20 vs. TasQ, dose response inhibition of the in vitro growth was tested in a series of human prostate cancer cell lines covering the clinical genotype/phenotype spectrum were utilized. This included the androgen receptor positive (AR+)/castration sensitive LNCaP, the AR+/castration resistant (CR) CWR-22Rᵥ1, the AR negative (AR−)/CR BCaP, and the AR−/CR LgCaP-1. In previous studies, the concentration of TasQ for inhibiting in vitro growth of human prostate cancer lines by 50% (i.e. IC₅₀ value) is ˜50 μM (Isaacs 2013). In contrast, ESATA20's IC₅₀ value is <10 μM (p<0.05) against 3 of the 4 lines (FIGS. 48A, 48B, and 48C). Only against the LNCaP line is ESATA20's IC₅₀ value >10 μM. Importantly, however when 1 OM of ESATA20 is combined with 3 nM docetaxel, LNCaP growth is completely inhibited (p<0.05) which does not occur when docetaxel is combined with 10 μM of TasQ (FIG. 48D). Importantly this enhancement of the taxane response in LNCaP cells by ESATA20 phenocopies the enhanced taxane response reported previously when HDAC4 is KD under a hypoxic condition (Geng 2011).

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the disclosure, may be made without departing from the spirit and scope thereof.

1. A compound of formula (I): 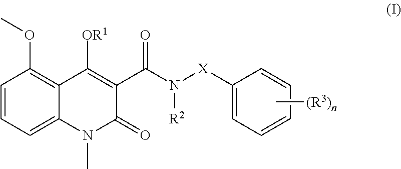
or a pharmaceutically acceptable salt thereof, wherein:
R¹ is selected from hydrogen and C₁-C₄ alkyl, C₁-C₄ hydroxyalkyl, and C₁-C₄ aminoalkyl, wherein the amino of the C₁-C₄ aminoalkyl is optionally protected by a protecting group;
R² is selected from hydrogen, C₃-C₁₀ alkyl, C₃-C₆ cycloalkyl, and arylalkyl, wherein the alkyl, cycloalkyl, and arylalkyl are each optionally substituted with 1, 2, or 3 substituents independently selected from halo, C₁-C₄ haloalkyl, hydroxy, C₁-C₄ alkoxy, and arylalkyloxy;
n is 0, 1, 2, or 3;
each R³ is independently selected from C₁-C₄ haloalkyl, halo, C₁-C₄ alkyl, and C₁-C₄ alkoxy; and
X is a bond or —N═CH—, wherein if X is a bond, R² is not hydrogen.

2. The compound of claim 1, or a pharmaceutically acceptable salt thereof, wherein R¹ is selected from hydrogen, methyl, ethyl, and 2-aminoethyl, wherein the amino of the 2-aminoethyl is optionally protected by a tert-butyloxycarbonyl group.

3. The compound of claim 1 or claim 2, or a pharmaceutically acceptable salt thereof, wherein R¹ is hydrogen.

4. The compound of any one of claims 1-3, or a pharmaceutically acceptable salt thereof, wherein X is a bond, and R² is selected from C₃-C₁₀ alkyl, C₃-C₆ cycloalkyl, and arylalkyl, wherein the alkyl, cycloalkyl, and arylalkyl are each optionally substituted with 1, 2, or 3 substituents independently selected from halo, C₁-C₄ haloalkyl, hydroxy, C₁-C₄ alkoxy, and arylalkyloxy.

5. The compound of claim 4, or a pharmaceutically acceptable salt thereof, wherein R² is selected from isopropyl, n-heptyl, cyclohexyl, benzyl, and ethyl substituted with one benzyloxy group.

6. The compound of any one of claims 1-3, or a pharmaceutically acceptable salt thereof, wherein X is —N═CH— and R² is hydrogen.

7. The compound of any one of claims 1-6, or a pharmaceutically acceptable salt thereof, wherein n is 0.

8. The compound of any one of claims 1-6, or a pharmaceutically acceptable salt thereof, wherein n is 1.

9. The compound of claim 8, or a pharmaceutically acceptable salt thereof, wherein R³ is substituted at the para position of the phenyl group.

10. The compound of claim 8 or claim 9, or a pharmaceutically acceptable salt thereof, wherein R³ is C₁-C₄ haloalkyl.

11. The compound of claim 10, or a pharmaceutically acceptable salt thereof, wherein R³ is trifluoromethyl.

12. The compound of claim 1, or a pharmaceutically acceptable salt thereof, having formula (Ia): 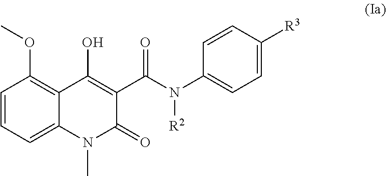
wherein:
R² is selected from C₄-C₆ alkyl and C₃-C₆ cycloalkyl; and
R³ is independently selected from C₁-C₄ haloalkyl, halo, C₁-C₄ alkyl, and C₁-C₄ alkoxy.

13. The compound of claim 12, or a pharmaceutically acceptable salt thereof, wherein R² is C₃-C₆ cycloalkyl, and R³ is C₁-C₄ haloalkyl.

14. The compound of claim 1, wherein the compound is selected from: 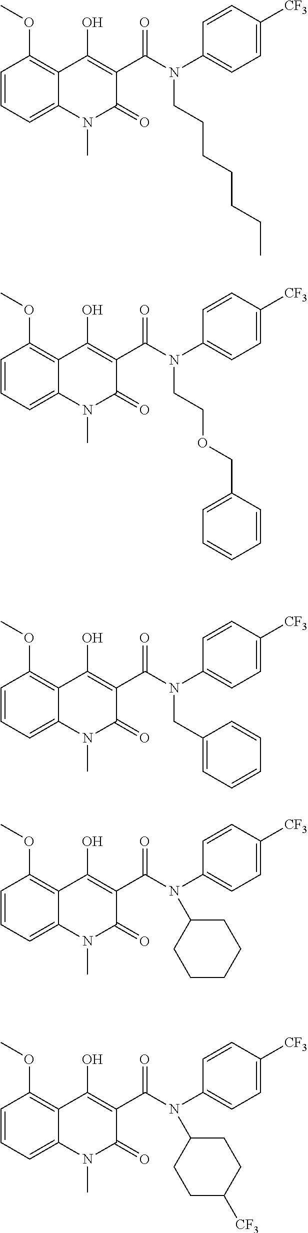 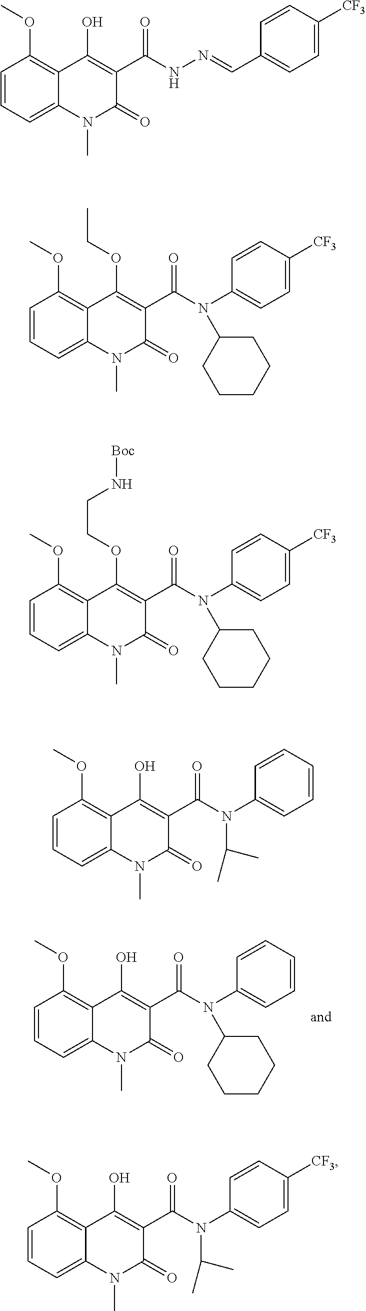 [SMILES: COc1cccc2c1c(O)c(C(=O)N/N=C/c1ccc(C(F)(F)F)cc1)c(=O)n2C ; CCOc1c(C(=O)N(c2ccc(C(F)(F)F)cc2)C2CCCCC2)c(=O)n(C)c2cccc(OC)c12 ; COc1cccc2c1c(OCCNC(=O)OC(C)(C)C)c(C(=O)N(c1ccc(C(F)(F)F)cc1)C1CCCCC1)c(=O)n2C ; COc1cccc2c1c(O)c(C(=O)N(c1ccccc1)C(C)C)c(=O)n2C ; COc1cccc2c1c(O)c(C(=O)N(c1ccccc1)C1CCCCC1)c(=O)n2C ; COc1cccc2c1c(O)c(C(=O)N(c1ccc(C(F)(F)F)cc1)C(C)C)c(=O)n2C]
or a pharmaceutically acceptable salt thereof.

15. A pharmaceutical composition comprising a compound of any one of claims 1-14, and a pharmaceutically acceptable carrier.

16. A method of treating cancer in a subject in need thereof, comprising administering to the subject a therapeutically effective amount of a compound of any one of claims 1-14, or a pharmaceutical composition of claim 15.

17. The method of claim 16, wherein the cancer is prostate cancer.

18. The method of claim 14, wherein the prostate cancer is metastatic castration-resistant prostate cancer.

19. The method of any one of claims 16-18, further comprising administering an additional chemotherapeutic agent to the subject.

20. The method of claim 19, wherein the additional chemotherapeutic agent is a taxane.

21. The method of any one of claims 16-20, wherein the subject is a human.

22. Use of a compound of any one of claims 1-14, or a pharmaceutical composition of claim 15, in the treatment of cancer.

23. The use of claim 22, wherein the cancer is prostate cancer.

24. The use of claim 23, wherein the prostate cancer is metastatic castration-resistant prostate cancer.
